(19) United States
(12) Patent Application Publication
(10) Pub. No.: US 2008/0009771 A1
(43) Pub. Date: Jan. 10, 2008

(54) Exoskeleton

(76) Inventors: Joel Perry, Bellevue, WA (US); Jacob Rosen, Seattle, WA (US)

Correspondence Address: SCHWEGMAN, LUNDBERG & WOESSNER, P.A., P.O. BOX 2938, MINNEAPOLIS, MN, 55402, US

(21) Appl. No.: 11/729,998

(22) Filed: Mar. 29, 2007

Related U.S. Application Data
(60) Provisional application No. 60/743,934, filed on Mar. 29, 2006.

(51) Int. Cl. A61B 5/103

(52) U.S. Cl. 600/587

(57) ABSTRACT

This document discloses, among other things, a wearable structure having links and joints corresponding to those of a human upper body. Transducers are located on the wearable structure and are coupled to a processor. The transducers exchange energy and information between the user and the wearable structure and enable control of the movement of the structure.

### CROSS-REFERENCE TO RELATED PATENT DOCUMENTS

This document claims the benefit of priority, under 35 U.S.C. Section 119(e), to Rosen, U.S. Provisional Patent Application Ser. No. 60/743,934, entitled “EXOSKELETON FOR PHYSICAL THERAPY,” filed on Mar. 29, 2006 (Attorney Docket No. 2082.009PRV), and is incorporated herein by reference.

### GOVERNMENT RIGHTS

This invention was made with Government support under Contract or Grant No. IIS0208468 awarded by National Science Foundation. The Government has certain rights in this invention.

### TECHNICAL FIELD

This document pertains generally to robotics, and more particularly, but not by way of limitation, to an exoskeleton.

### BACKGROUND

Previous attempts to build a powered exoskeleton have been inadequate for various reasons. In some cases, the processor and control algorithms were too slow to make the structure move naturally with the user. In others, the power supplies and actuators have been cumbersome and sluggish.

### OVERVIEW

The present systems and methods relate to an exoskeleton, or a wearable robot having joints and links corresponding to those of the human body. The system and method can be used in rehabilitation medicine, virtual reality simulation, and teleoperation, and for the benefit of both disabled and healthy populations.

The present system includes an anthropomorphic, seven degree-of-freedom, powered upper body exoskeleton. One example includes proximal placement of drive motors and distal placement of cable-pulley reductions, thus yielding low inertia, high-stiffness links, and back-drivable transmissions with zero backlash. One example enables full glenohumeral, elbow, and wrist joint functionality.

The human-machine interface is established at the neural level based on a Hill-based muscle model (myoprocessor) that enables intuitive interaction between the operator and the wearable robot. Some potential applications of the exoskeleton include an assistive (orthotic) device for human power amplifications, a therapeutic and diagnostics device for physiotherapy, a haptic device for use in virtual reality simulation, and a master device for teleoperation.

The exoskeleton of the present subject matter includes an external structural mechanism with joints and links corresponding to those of the human body. When used as an assistive device, the human wears the exoskeleton, and its actuators generate torques applied on the human joints. When used as a human power amplifier, the human provides control signals for the exoskeleton while the exoskeleton actuators provide some of the power necessary for task performance. The human becomes part of the system and applies a scaled-down force in comparison with the load carried by the exoskeleton. When used as a master device in a teleoperation system, the operator controls a secondary, possibly remote, robotic arm (slave). In a bilateral mode, the forces applied on the remote robotic arm by the environment are reflected back to the master and applied to the operator's arm by the exoskeleton structure and actuators. In this configuration, the operator feels the interaction of the robotic arm tool-tip with the environment. When used as a haptic device, the present subject matter enables human interaction with virtual objects simulated in virtual reality. As a result, virtual objects can be touched by the operator. The exoskeleton structure and its actuators provide force feedback, emulating the real object including its mechanical and textural properties. The exoskeleton, in that sense, simulates an external environment and adds the sense of touch (haptics) to the graphical virtual environment. Several mechanisms including arms, hands, legs and other haptic devices can be used.

This overview is intended to provide an overview of the present subject matter. It is not intended to provide an exclusive or exhaustive explanation. The detailed description is included to provide further information about the present subject matter.

### BRIEF DESCRIPTION OF THE DRAWINGS

In the drawings, which are not necessarily drawn to scale, like numerals may describe substantially similar components in different views. Like numerals having different letter suffixes may represent different instances of substantially similar components. The drawings illustrate generally, by way of example, but not by way of limitation, various embodiments discussed in the present document.

FIG. 1 illustrates a block diagram of one example of the present subject matter.

FIG. 2 illustrates angular variations between elbow flexion-extension and pronosupination axes resulting in different elbow flexion kinematics.

FIG. 3 illustrates joint axes for a human.

FIGS. 4A, 4B and 4C illustrate various joint configurations.

FIG. 4D illustrates an exoskeleton.

FIG. 5 illustrates a model of exoskeleton axes in relation to a human arm.

FIG. 6 illustrates some exoskeleton configurations that achieve rotation about the long axis of a limb segment.

FIG. 7 illustrates mechanical singularities.

FIG. 8 illustrates a two stage reduction drive.

FIG. 9 illustrates cable routing.

FIG. 10 illustrates a system block diagram and feedback control loops.

FIG. 11 illustrates a low level block diagram of a Hill-based muscle model.

FIG. 12 includes a block diagram of an algorithm for evaluating neural activation level based on sEMG.

FIG. 13A illustrates exoskeleton operation in virtual reality with a user wearing a head mounted display.

FIG. 13B illustrates rendered workspace of the upper limb with a limited range of motion of the joints.

FIG. 14 illustrates a block diagram of a system according to one example.

FIGS. 15A and 15B illustrate perspective views of a model human wearing an exoskeleton.

FIGS. 16A and 16B illustrate perspective views of an exoskeleton.

### DETAILED DESCRIPTION

The following detailed description includes references to the accompanying drawings, which form a part of the detailed description. The drawings show, by way of illustration, specific embodiments in which the invention may be practiced. These embodiments are also referred to herein as “examples.” The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one, independent of any other usages of “at least one” or “one or more.” In this document, the term “or” is used to refer to a nonexclusive or, such that “A or B” includes “A but not B,” “B but not A,” and “A and B,” unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

Introduction

In one example, the human-machine interface (HMI) is positioned at the neuromuscular level, a relatively high level of human physiological (neurological) system hierarchy, in order to reduce the effects of the electro-chemical-mechanical delay. The electro-chemical-mechanical delay, usually referred as the electro-mechanical delay (EMD), refers to the interval between the time when the neural system activates the muscular system and the time when the muscles and the associated soft tissues mechanically contract and generate moments around the joints. By establishing the interface at the neuromuscular level, the present subject matter can estimate the forces that will be generated by the muscles, using a muscle model, before the muscle contraction actually occurs. As a result, the reaction time of the human/machine system is reduced, resulting in a more natural control of the task. In line with this concept, an exoskeleton is disclosed in which the HMI is set at the human neuromuscular junction.

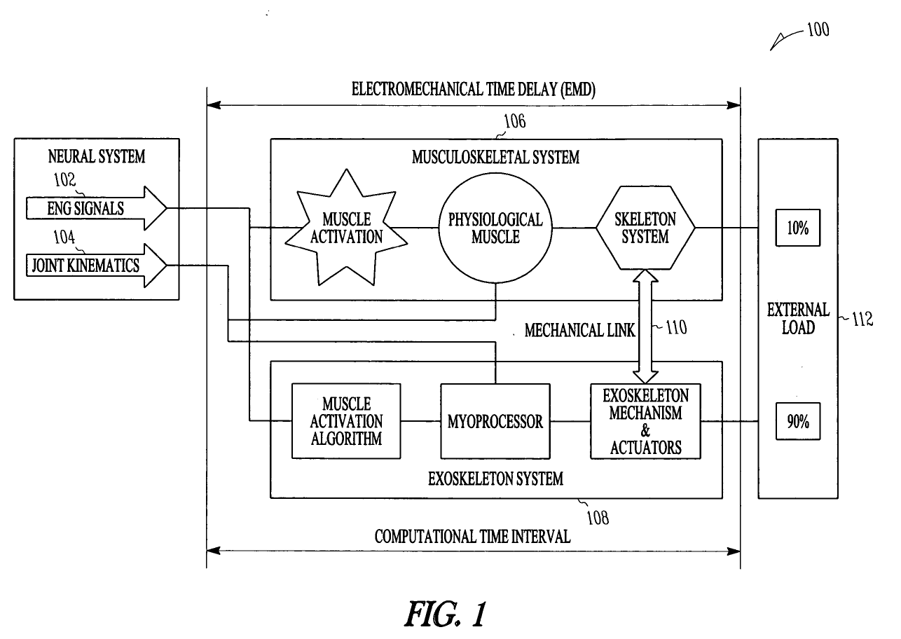

The HMI of the present subject matter is positioned at the neuromuscular level and uses processed surface electromyographic (sEMG) signals as a command signal of the system as shown in FIG. 1. These signals are the same signals initiated by the human's central nervous system to contract the human's own actuators (the muscles). As such, this example uses a myoprocessor to control the structure.

FIG. 1 illustrates a block diagram of one example of the present subject matter. In system 100 illustrated in the figure, the input includes EMG signals 102 and joint kinematic data 104. The user's musculature system 106 and the exoskeleton 108 are in parallel alignment and coupled by mechanical link 110. The load 112 is shared by the parallel elements.

During the EMD, the system gathers information regarding the physiological muscle's neural activation level based on processed EMG signals, the joint position, and angular velocity. This information is provided to the myoprocessor which in turn predicts the moment that will be developed by the physiological muscle relative to the joint. The predicted moment is provided to the exoskeleton system such that, by the time the physiological muscle contracts, the exoskeleton amplifies the joint moment by a preselected gain factor. Part of the time gained by using these predicted muscle moments is employed by the electromechanical subsystems of the powered exoskeleton to compensate for their own inherent reaction time.

The upper limb includes segments linked by articulations with multiple degrees of freedom and is able to perform tasks involving both power and precision of movement. A lower limb exoskeleton system may also include control for balance and dynamic components of gait to allow standing and walking.

An electromechanical system may be fully portable or stationary. A portable system may be limited by the power to weight ratio of the power source. For the human upper limb, the exoskeleton system can be part of a stationary working station or fixed to the frame of a powered wheelchair and powered by the wheelchair battery or other power supply.

Setting the HMI at the neuro-muscular level may lead to seamless integration and intuitive control of the exoskeleton arm as a natural extension of the human body. One component of the exoskeleton HMI includes a model of the human muscle, the myoprocessor, running in real-time and in parallel to the physiological muscle, that predicts joint torques as a function of the joint kinematics and neural activation levels. One example of the present subject matter includes one or more myoprocessors for the upper limb based on the Hill phenomenological muscle model. In one example, a genetic algorithm is used to configure the internal parameters of the myoprocessors utilizing an experimental database that provides inputs to the model and allows for performance assessment.

Previous exoskeleton designs have primarily utilized internal-external rotation joints and prono-supination joints that fully enclose the arm, requiring the user to enter the exoskeleton from the device shoulder and slide his/her arm axially down the length of the device through the closed circular bearings. This can be a difficult and even uncomfortable task for users depending on the severity of impairment. In the current exoskeleton, the use of open mHMI's for both upper and lower arm segments eliminates this difficulty.

Integrating human and robotic-machines into one system offers multiple opportunities for creating a new generation of assistive technology for both healthy and disabled people. For many physical tasks, human performance is limited by muscle strength. In addition, muscle weakness is the primary cause of disability for most people with neuromuscular diseases, including stroke, spinal cord injury, muscular dystrophies, and other neuro-degenerative disorders. In contrast to this strength limitation, humans possess specialized and complex algorithms for control of movement, involving higher and lower neural centers, that enable them to perform very complicated tasks such as locomotion and arm movement, while at the same time avoiding object collisions. In contrast, robotic manipulators can be designed to perform tasks requiring large forces or moments, depending on their structure and on the power of their actuators. However the control algorithms which govern their dynamics lack the flexibility to perform in a wide rage of conditions while preserving the same quality of performance as humans. Combining these two entities, the human and the robot into one integrated system under the control of the human, can benefit from the advantages offered by each subsystem. The mechanical power of the machine, integrated with the inherent human control system, could allow efficient performance of tasks requiring higher forces than the human could otherwise produce.

The HMI can be set at the neuromuscular level using processed sEMG signals initiated by the human's central nervous system to contract the human's own actuators—the muscles, as the primary control signal to the exoskeleton. As opposed to neural prostheses that provide control using EMG signals as simple on/off switches, with assistance from visual feedback, the exoskeleton device incorporates more complex control algorithm. This provides a more natural operation of the device, due to the high level of synergism resulting from the operator arm being in full contact with the exoskeleton.

The present subject matter enables prediction of the force generated by the muscle solely based on processed EMG signals. Such predictions are made in an isometric condition (static conditions). In dynamic conditions where the muscle is changing length and velocity, the situation is different.

The present subject matter leaves the EMG signal in its original domain as a neural signal. Using a signal processing algorithm, the neural activation of the muscle is predicted from the raw EMG signals. In addition to the neural activation levels, the myoprocessor takes into account the joint's kinematics to predict the muscle force and the moment applied on the joint. The myprocessor prediction is essentially the command signal to the hard core control system.

### I. PRELIMINARIES

Performance

A quantitative measure of system performance is bandwidth. Systems having a higher bandwidth are controllable under higher frequency command signals. Limited by the system's lowest natural frequency, the bandwidth is a measure of how success as to the trade-offs between weight and stiffness.

In one example of the present subject matter, the bandwidth is 10 Hz based on actual weights of 3.5 kg and 6.3 kg for link 1 and links 2-7, respectively.

In various examples of the present subject matter, the exoskeleton is controlled based on a level of the human machine interface (HMI) between the exoskeleton robot and the human operator described as: (I) kinematic; (II) dynamic; (III) neuromuscular, e.g., surface electromyography (sEMG); (IV) brain, e.g., noninvasive electroencephalogram (EEG) or invasive action potential signal measured directly from the motor cortex.

At the neuromuscular level, the body's own neural command signals are used as command signals of the exoskeleton. By establishing the interface at the neuromuscular level, the effects of muscle contractions can be estimated before these effects can be directly measured using other means (e.g., kinematic and dynamic interfaces). An electro-(chemical)-mechanical delay (EMD) inherently exists in the musculoskeletal system and this time delay refers to the interval between the time when the neural system activates the muscular system and the time when the muscles and the associated soft tissues contract mechanically and generate moments around the joints. EMD values vary considerably depending on the muscle, the person, and the experimental technique used for the measurements and can be assumed to be in the range of 26-131 ms with values for some upper limb muscles in the middle-lower part of this interval.

A. Physical (Mechanical) Human-Machine Interface(s)

The physical components that mechanically couples the human arm and the exoskeleton structure, and enable force transmission between them are referred to as the mechanical HMI (mHMI). The mHMI can be tailored to different users based on the level of muscular and functional impairment or other factors.

B. Safety Considerations

In various examples, safety precautions are implemented at the mechanical, electrical, and software levels.

At the mechanical (hardware) level, physical stops prevent segments from excessive excursions that could hyperextend or hyperflex individual joints of the user. Also, pulleys in some joints are driven purely by friction. This allows the transmission to slip if the force between user and device exceeds a set limit. In one example, brakes are provided on all actuators. Electromechanical brakes are used with all servo actuators. The brakes are design to overcome the maximal mechanical torques generated by the actuators. Engaging the brakes stop the movements of all the servo system at once, regardless of the inputs provided to the servo system. As a result the system freezes and maintains the arm position in space.

One example includes physical joint limits. As such, the Exoskeleton mechanism includes physical joint limits constraining the range of motion of each joint to the physiological/anatomical range of motion. These physical stops prevent any potential joint dislocation.

At the electrical level, the system is equipped with three emergency shutoff switches: an enable button that terminates the motor command signal upon release, a large e-stop button for complete power shutoff by the observer, and a similar e-stop foot switch for the user.

One example uses redundant position sensing. The joint position of each DOF is sensed by two sensors: a shaft encoder located on the actuator and potentiometer located on the exoskeleton joint. This redundant setup of sensors allows triggering an E-stop in any case that a discrepancy between the readout of the two sensors will be identified. Such a discrepancy may occur whenever one of the sensors fails or any damage to the structure element takes place such as mechanical cable breakout or mechanical deformation of the link.

One example uses an emergency stop or “E-stop.” An E-stop is a state of the system that can be triggered both by hardware and software. One example of the system includes three E-stop buttons that can be activated by the user, the exoskeleton operator or the therapist. Upon pressing the E-stop button, the power to the servo amplifiers is disconnected and the brakes are engaged. In addition software based E-stop triggers the same response to the hardware trigger base on sensors' discrepancy and internal logic that is incorporated into the system (e.g. position sensors miss-match, exceeding operational envelop).

At the software level, one example of the system includes redundant position sensors (potentiometer—Midori, Fullerton; shaft encoder—HP), with one sensor each at either end of the power train to monitor both joint motion and motor position. Redundant position sensing enables the software to monitor power transmission integrity; any slip occurring between the motors and end-effector will result in a position discrepancy and lead to immediate system shut-down. Software limits are also implemented on commanded motor currents, (for example, motor torques).

One example includes position/velocity/acceleration limits. These thresholds on the position, velocity and acceleration of the joints are implemented into the control software. These limits gradually increase over time based on the operator conditions up to values associated with a controlled movement of a normal subject. The system will freeze (e-stop) if the operational value will reach a safeguard margin of 5% of the selected limits.

One example includes force limits. Force limits are thresholds on the interaction forces between the exoskeleton and the operator that are implemented into the control software. High interaction forces may develop if the exoskeleton moves to the opposite direction of the operator or when the movement exceeds the workspace of the operator. These limits will gradually increase based on the operator conditions up to values associated with controlled movements of a normal subject. The system will freeze (e-stop) if the operational value will reach a safeguard margin of 5% of the selected limits.

One example includes virtual fixtures. These are thresholds on the range of motion of each joint which are smaller than the normal range of motion implemented in software. The exoskeleton will stop once the joint angle reaches its limit. Any application of force as an attempt to exceed this limit will trigger the force limit and will result in an e-stop. The joint range of motion will be gradually increased based on the operator conditions up to the maximal physical joint limits that are incorporated into the hardware of the system.

One example includes gravity compensation. Gravity compensation is the ability of the system to support its own weight as well as the weight of the operator's arm and hand. The gravitational compensation implementation emulates arm and hand movements in a zero gravity environment. The joint torque for compensating the gravitational loads are calculated base on a dynamical model running in real time. This algorithm calculates the required joint torques base on the joint position and the anthropometrical information of the patient arm. Based on this calculation a set of commands is sent to the exoskeleton actuators (servo DC motors) utilizing a feed-forward control. In one example, the joint torques generated by the 7 actuators always supports the gravitational loads that are generated by the exoskeleton arm itself however the extent in which the patient's arm is supported can be adoptive based on the experimental protocol and recovery of the subject. The present subject matter enables adapting the gravitational field for different circumstances. Gravity compensation as a mode of operation can affect treatment in a therapeutic application. The gravitational field can be gradually introduced as the treatment is progressing by gradually decreasing the compensation. However, gravity compensation can also be used as a safety precaution allowing the patient with limited muscle strength to explore the entire reachable workspace without exposure to gravitational loads which often exceed the muscle strength of the disabled operator and cause pain in proximal joints.

C. Modeling the Human

Anthropomorphic joint approximations can be modeled at varying degrees of accuracy and complexity. The level of complexity for a suitable representation depends on the desired tasks to be performed and replicated using the model. Shoulder motion, for example, including a glenohumeral (G-H), acromioclavicular, and sternoclavicular articulations, can be represented largely by the G-H joint for a variety of arm activities involving up to 90 degrees of arm elevation. With minimal activity exceeding this range, a simplified model of the shoulder may be appropriate. The G-H movement can further be simplified to a ball and socket joint having three orthogonal axes intersecting at the center of the humeral head, although the true center of rotation may vary with arm orientation. Rotations about these orthogonal axes can be treated as Euler rotations. The order of flexion-extension and abduction-adduction about the first two axes is arbitrary but should be noted, while the third rotation corresponds to internal-external rotation.

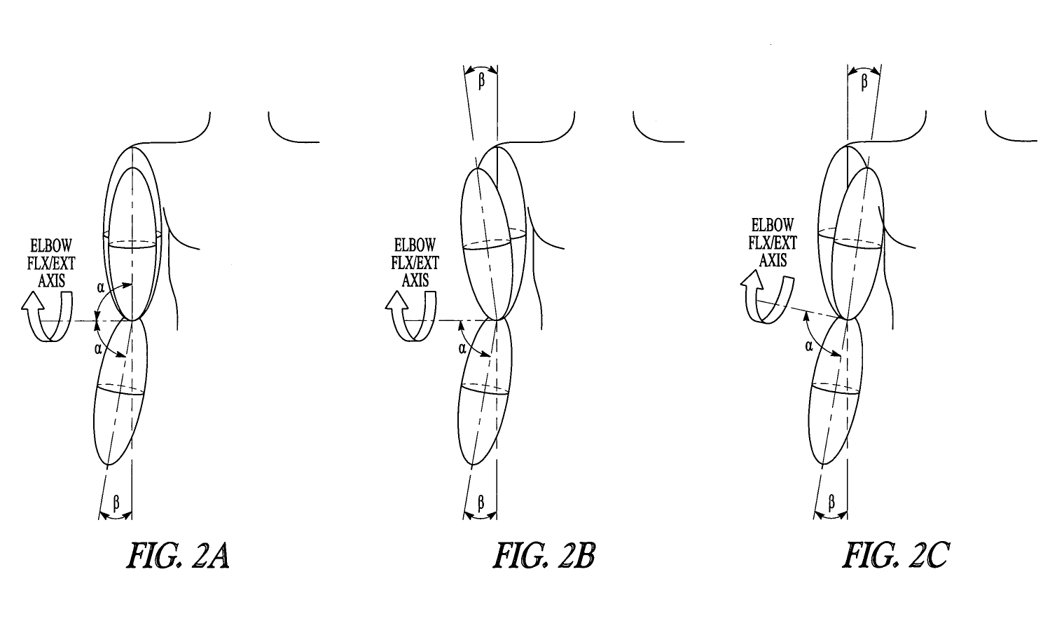

The elbow can be represented as a single-axis hinge joint where the hinge rests at an oblique angle with respect to both upper and lower arm segments under full arm extension as shown in FIGS. 2A, 2B, and 2C.

FIG. 2 illustrates angular variations between elbow flexion-extension and pronosupination axes resulting in different elbow flexion kinematics. FIG. 2A illustrates a Type I elbow having a symmetric axis with respect to both upper and lower arm segments. FIG. 2B and FIG. 2C illustrate less common examples.

Of the three elbow types, Type I (shown in FIG. 2A) is relatively common and is used in one example. The hinge offset accounts for lateral deviation of the forearm during supinated activities. Under full elbow extension and forearm supination, angular differences, β, of up to 10 degrees exist between the midlines of the upper and lower arm segments, and decrease with pronation. In one example, an assumed offset of zero degrees yields sufficient results and significantly reduced complexity of the resulting dynamic equations of motion.

Pronosupination of the forearm can be treated interchangeably as a freedom of the elbow and as a freedom of the wrist. In either case, it is considered directly adjacent to the forearm, occurring after elbow flexion and before either wrist flexion or deviation, with the axis of rotation running approximately through the 5th metacarpal-phalangeal joint.

The wrist can be modeled as two orthogonal axes with a fixed offset between them. The proximal and distal axes of the wrist correspond to wrist flexion-extension and wrist radial-ulnar deviation, respectively.

Although, anthropometrically, the wrist could be more accurately represented incorporating a slight offset between the flexion-extension and radial-ulnar deviation axes, this offset was neglected for simplicity. Unlike the neglected forearm offset, β, which was unnoticeable to the user, the high sensitivity of the wrist joint to changes in position and torque make this human-machine discrepancy mildly noticeable.

For one example of the present subject matter, the anatomic joints of the human upper limb are defined as ball/socket type of joints. In order to meet the requirements of an anthropomorphic joint design for the exoskeleton, the line of rotation of the exoskeleton joints coincide with the anatomical axis. For example in the shoulder joint, the two orthogonal rotation axes cross each other at a virtual point where the humeral head is rotating relative to the glenoid fossa (socket of shoulder joint). In addition, the third axis of rotation lies along the humerus bone allowing the anatomic motion of internal and external rotation. The length of the exoskeleton links can be adjustable to accommodate differences in the anthropomorphic arm dimensions. Moreover, in order to prevent injuries to the operator's joints, such as joint dislocation, the anatomical joints' range of motion is incorporated into the design of the exoskeleton joints. This assures that the range of motion of the exoskeleton joints will never exceed the range of motion of the operator.

A 7-DOF model of the human arm includes three segments (upper arm, lower arm and the hand) connected to each other and the human trunk with a frictionless ball-and-socket shoulder joint, 2-axis elbow and 2-axis wrist. Using this structure, 7 equations of motion can be written. The mass, the center of mass location, and the inertia of the human arm segments can be estimated for each subject. The general form of the equations of motion is expressed in Equation 1.
τ=M(Θ)\ddot{Θ}+V(Θ,\dot{Θ})+G(Θ) (1)
where M(Θ) is the 7×7 mass matrix, V(Θ,\dot{Θ}) is a 7×1 vector of centrifugal and Coriolis terms, G(Θ) is a 7×1 vector of gravity terms, and τ is a 7×1 vector of the net torques applied at the joints. Given the kinematics ofthe human arm (\ddot{Θ},\dot{Θ},Θ) the individual contributions on the net joint toque (τ) vector can be calculated for each action of each subject. The net torque can be calculated from the contribution of the individual components (inertial, centrifugal, and Coriolis, and gravity).

Various muscle models exist that provide a quantitative representation of contraction dynamics. Such models differ in intended application, mathematical complexity, level of structure considered and fidelity to the biological facts. Some muscle models include 1) microscopic models; 2) distributed moment models; 3) macroscopic models; 4) fiber models. Hill-based models, viscoelastic models, and system models are subcategories of the macroscopic models class.

In various examples of the present subject matter, the muscle is modeled using two macroscopic muscle models including a Hill-based muscle model and neural network model as part of a HMI for a single degree-of-freedom (DOF) powered exoskeleton.

During certain positioning tasks, higher angular velocities are observed in the gross manipulation joints (the shoulder and elbow) as compared to the fine manipulation joints (the wrist). An inverted phenomenon is noted during fine manipulation in which the angular velocities of the wrist joint exceeded the angular velocities of the shoulder and elbow joints. Analyzing the contribution of individual terms of the arm's equations of motion indicate that the gravitational term is the most dominant term in these equations. The magnitudes of this term across the joints and the various actions is higher than the inertial, centrifugal, and Coriolis terms combined. Variation in object grasping (e.g. power grasp of a spoon) alters the overall arm kinematics in which other joints, such as the shoulder joint, compensate for lost dexterity of the wrist. The collected database along with the kinematics and dynamic analysis provide the fundamental understanding for designing the powered exoskeleton for the human arm as well as the effect of joint compensations in case of disability.

It has been stated that to achieve proper correlation between Euler angle model representations and the actual biomechanics of the arm, forearm pronosupination should precede both wrist flexion and deviation axes.

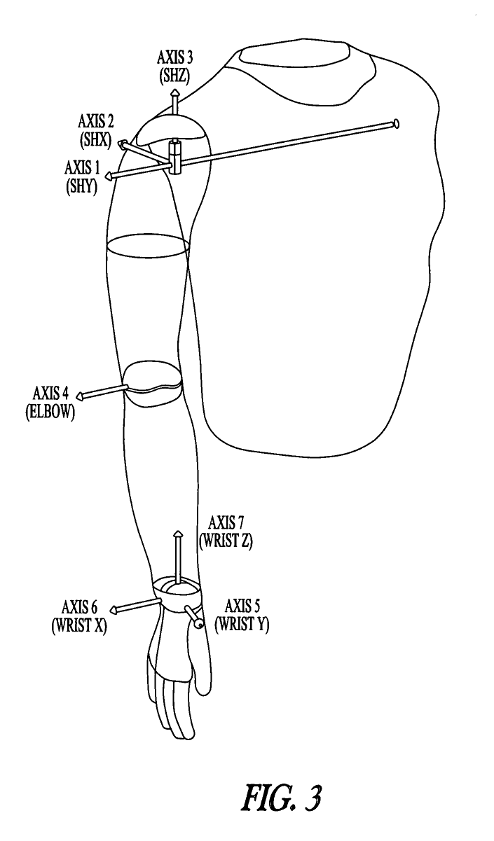

The Vicon axes designated in FIG. 3 as axes 5, 6, and 7 cannot be directly compared to anatomical motions of wrist flexion, deviation, and rotation. They can instead be considered as a set, with axes 5 and 6 only corresponding to flexion-extension and radial-ulnar deviation when pronosupination is near zero, and axis 7 only corresponding to pronosupination when both wrist flexion and deviation are near zero.

In general, the largest ranges of motion during daily tasks are found in elbow flexion-extension and forearm pronosupination, each at 150 degrees, while the requirement from shoulder flexion-extension, the joint having the largest physiological range of motion, remains less at 110 degrees. Average joint torques seen in the elbow and wrist are approximately one tenth and one one-hundredth, respectively, of those experienced at the shoulder, with median torques at the shoulder ranging from 0.4 to 4 Nm.

### II. EXOSKELETON STRUCTURE

This section describes the skeleton (orthotic device) mechanism itself and its biomechanical integration with the human body

The present subject matter can be configured to have a variety of degrees of freedom. For example, the system can be configured for 1-DOF, 3-DOF, or 7-DOF. In various examples, system is controlled based on surface electromyographic (sEMG) signals. In the case of a 1-DOF system, for example, bicep and tricep sEMG signals can be used to command the elbow joint torque and in the case of a 3-DOF system, two additional degrees are added at the shoulder level, thus incorporating additional sEMG command signals from shoulder muscles.

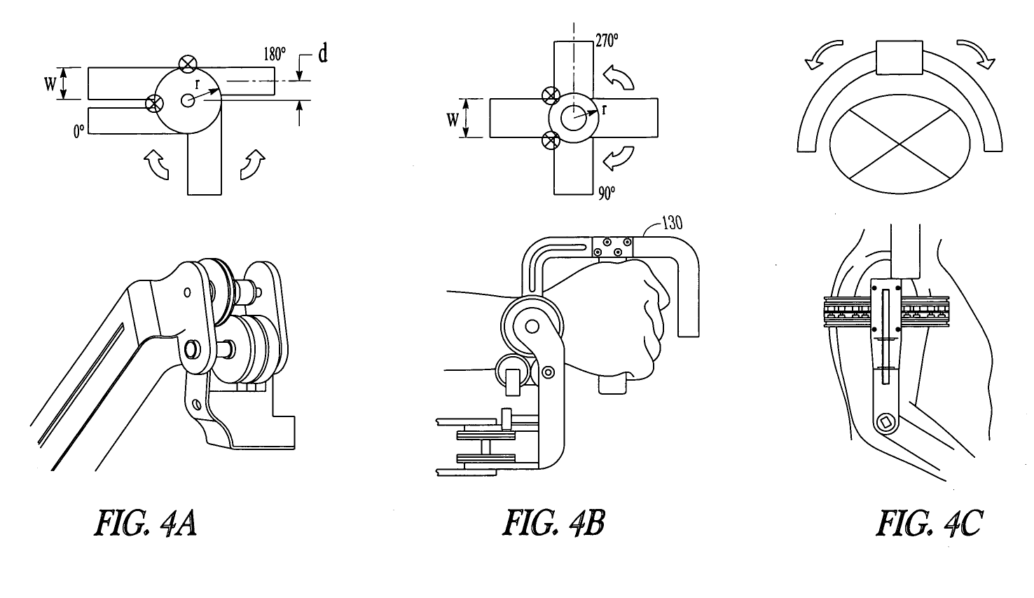

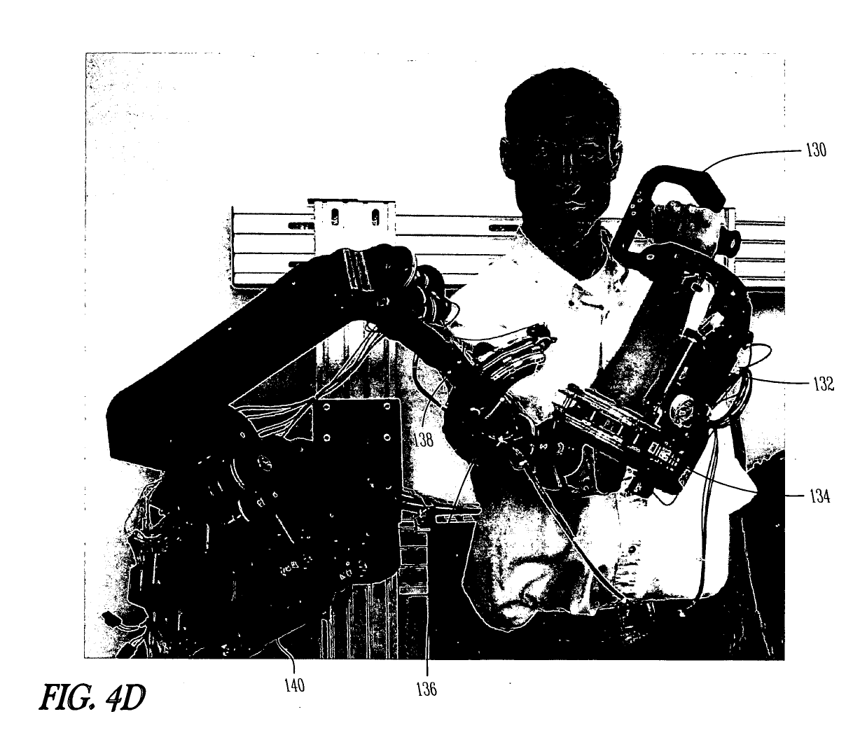

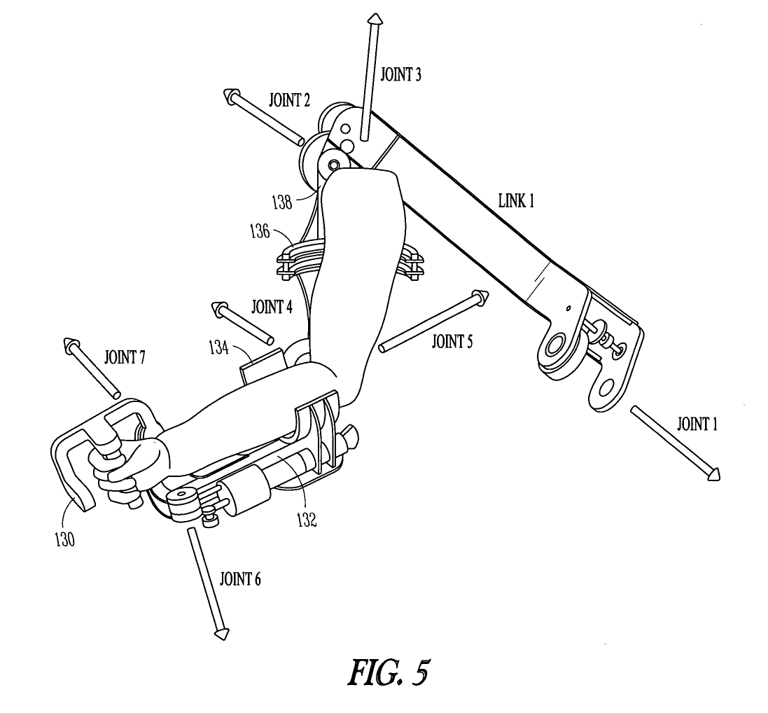

An example of the exoskeleton arm includes seven degrees of freedom. The exoskeleton arm is actuated by seven DC brushed motors (Maxon) that transmit torque to each joint utilizing a cable-based transmission system. Four force/torque sensors (ATI-Nano 17) are located at all interface elements between the human arm and exoskeleton as well as between the exoskeleton and external load, measuring all forces and torques acting and reacting between the human arm, the external load, and the exoskeleton, as shown in FIG. 4D. Elements illustrated in the figure include hand piece 130, lower arm link 132, circular bearing 134 for the lower arm, circular bearing 136 for the upper arm, upper arm link 138, and actuators 140.

FIGS. 4A, 4B and 4C illustrate various joint configurations used in the exoskeleton of FIG. 4D. The exoskeleton of FIG. 4D includes various joints that achieve full glenohumeral, elbow, and wrist functionality. FIG. 4B includes an illustration of hand piece 130.

In one example, each force/torque sensor is a 6-axis sensor. In various examples, the sensor is a silicon strain gauge or a foil gauge. The exoskeleton is attached to a frame mounted on a wall, which allows adjustment of height and adjustment of the distance between the arms.

Articulation of the exoskeleton is achieved about seven single-axis revolute joints: one for each shoulder abduction-adduction (abd-add), shoulder flexion-extension (flx-ext), shoulder internal-external (int-ext) rotation, elbow flx-ext, forearm pronation-supination (pron-sup), wrist flx-ext, and wrist radial-ulnar (rad-uln) deviation. The exoskeletal joints are labeled 1 through 7 from proximal to distal in the order shown in FIG. 5. FIG. 5 illustrates a model of exoskeleton axes in relation to a human arm. Positive rotations about each joint produce the following motions: 1) combined flx/abd, 2) combined flx/add, 3) int rotation, 4) elbow fix, 5) forearm pron, 6) wrist ext, and 7) wrist rad dev. Note that the order and orientation of some joints are different from the axes presented in FIG. 3. Elements illustrated in FIG. 5 include hand piece 130, lower arm link 132, circular bearing 134 for the lower arm, circular bearing 136 for the upper arm, upper arm link 138, and actuators 140.

In one example, the exoskeleton joints are aligned with those of the human user. In particular, the rotational axis of the exoskeleton joint is aligned with the anatomical rotation axes. If more the one axis is involved at a particular anatomical joint (for example, the shoulder and the wrist), the exoskeleton joints emulating the anatomical joint intersect at the center of the anatomical joint. The glenohumeral (G-H) joint is modeled as a spherical joint having three intersecting axes. The elbow is modeled by a single axis orthogonal to the third shoulder axis, with a joint stop preventing hyperextension. Exoskeletal prono-supination takes place between the elbow and wrist joints as it does in the physiological mechanism. Two intersecting orthogonal axes represent the wrist. The range of motion of the exoskeleton joints support the ranges of motion encountered in most daily activities.

In the present subject matter, the human arm occupies a relatively large volume along the joint axis of rotation. This volume is to remain clear of obstacles and component configurations that could result in injury or discomfort to the user. Semi-circular bearings are used to allow users to don the device without strain or discomfort. The link adjacent to these semi-circular axes carries the mechanical components of the HMI. The mechanical HMI (mHMI) includes a pressure-distributive structural pad rigidly mounted to a six-axis force/torque sensor and is simultaneously securely fastened to the mid-distal portion of each respective arm segment. The fastening mechanism can include one or more straps made from one or more material (nylon, neoprene, metal), one or more wraps, or can include attachable or movable rigid components that partially or fully enclose the arm segment.

Those mHMI's that are placed high on the arm may produce unnaturally high forces through the interface points when operated in an assistive mode. In addition, compliance of the musculature in the proximal regions of the limb may contribute to compliance in the attachment, as well as variations in circumference potentially leading to further user discomfort. Cross-sections at distal parts of the limb segments are less variable in magnitude and thus experience reduced underlying skeletal transformation, making these locations better suited for mHMI attachment.

The exoskeleton includes an external structural mechanism with joints and links corresponding to those of the human body. In the case of the 7 DOF mechanism it includes 3 DOF for the shoulder joint 2 DOF for the elbow joint, and 3 DOF for the wrist joint (FIG. 4). The anthropometrical mechanism has a reachable workspace that overlap the workspace of the human arm. The operator who wears the exoskeleton can reach any point in space that is reachable without it. As a powered mechanism, 7 actuators are incorporated into its structure supporting the 7 degrees of freedom. Four out of the seven actuators are located on a stationary base supporting the three degrees of freedom of the shoulderjoint as well as the flexionlextension of the elbow joint. Three actuators are located on the exoskeleton forearm actuating the forearm rotation as well as the two degrees of freedom of the wrist joint. The mechanism as a whole is actuated through cables transmitting the required torques from the actuators to each of the joints. The cable actuated approach allows some of the actuators to be placed in a stationary base such that the weight and inertia of the mechanism are minimized providing backdrivability and superior dynamics to the operator. Four multi-axes force sensors are located in all the human machine interfaces: handle, lower arm, upper arm and the tip of the mechanism where it interacts with the environment. A redundant sets of position sensors are distributed along the manipulator. Each joint (DOF) has two position sensors one (potentiometer) incorporated into the joint itself and another one (optical encoder) located on each one of the DC motors. A 32 channels EMG amplifier as well as 64 surface electrodes with various contact surface are incorporated into the system. The system is under the control of 2 PCs one of which is simultaneously used for real-time servo control as well as data acquisitions.

Anatomically, the lower arm (also referred to as the forearm, between the wrist and elbow) is able to twist along it's length in a manner that can be modeled in the exoskeleton by ajoint located at the elbow or ajoint located at the wrist. This joint, in the present subject matter corresponds to the 5^{th} degree of freedom. According to the exoskeleton of the present subject matter, the position of the mHMI depends on the choice of locations for the joint. If the elbow joint is modeled with a single degree of freedom and the wrist is modeled with three degrees of freedom, then the forearm mHMI is located near the elbow. If the elbow joint is modeled with two degrees of freedom and the wrist is modeled with two degrees of freedom, then the forearm mHMI is located near the wrist. Other configurations are also contemplated.

A. Joint Design

Articulation of the exoskeleton is achieved about seven single-axis revolute joints: one for each shoulder abduction-adduction (abd-add), shoulder flexion-extension (flx-ext), shoulder internal-external (int-ext) rotation, elbow flx-ext, forearm pronation-supination (pron/sup), wrist flx-ext, and wrist radial-ulnar (rad-uln) deviation. The exoskeletal joints are labeled 1 through 7 from proximal to distal in the order shown in FIG. 5. Joint orientations are further addressed in another portion of this document.

B. Anthropomorphic Joints

In one example of an exoskeleton, three joint configurations are used. The configurations can be classified as 90-degree, 180-degree, or axial.

The joints are classified based on the relative alignment of adjoining links when the joint is approximately centered within its range of motion. Some joints of the body articulate about their mid-ROM when adjoining links are near orthogonal as illustrated in FIG. 4A. Other joints articulate about their mid-ROM when the links are near parallel, as in FIG. 4B. A third joint configuration relates to axial rotation of both the upper and lower arm segments, as illustrated in FIG. 4C. As shown in FIG. 4D, exoskeleton joints 1 and 7 are modeled as 180-degree joints, joints 2, 4, and 6 are 90-degree joints, and joints 3 and 5 are axial joints. Joint ROM for 90-degree configurations and 180-degree configurations can be increased either by increasing the central radius r, or by decreasing the link width w, as in FIG. 4A. Adjusting the link offset distance d, shifts the joint limits as illustrated by circles, and is effective to ‘tune’ the joint's mid-ROM.

The shoulder complex is reduced to a spherical joint having three axes intersecting at the center of the G-H joint. The elbow is modeled by a single axis orthogonal to the third shoulder axis. A joint stop prevents the joint from hyperextension. Exoskeletal prono-supination takes place midway between the elbow and wrist joints as it does in the physiological mechanism. Two intersecting orthogonal axes are used to represent the wrist.

C. Links and their Elements

In one example, some of the links are fabricated of “I-beam” channels having a joint located at each end. The links can be fabricated of various materials, including metal (such as aluminum, magnesium, or titanium) or non-metals (such as polymers, graphite or fiberglass).

In various examples, the links are coupled to the user's limb by fitted brackets, supports or other members that are shaped and sized to fit the user. The limbs can be attached using clamps, buckles, hook-and-loop fasteners, or other devices that securely hold the limb to the link.

D. Human-Machine Interface(s) (Sensors/Transducers)

Implementation of the axial joint configuration is complicated by the human arm occupying the joint axis of rotation, as represented by the elliptical shape in FIG. 4C. Occurring in axial rotations of both the upper and lower arm, the exoskeleton mHMI uses a semi-circular bearing design to allow users to don the device without strain or discomfort, as shown in FIG. 5. The semi-circular guides include three 60-degree curved-rail-bearing segments.

The mHMI's are the physical components that mechanically couple the human arm and the exoskeleton structure and enable force transmission between them. The interface is configured to be easily attached to the user. For patients of stroke and cervical spine injury, unassisted elevation of the arm is difficult if not impossible.

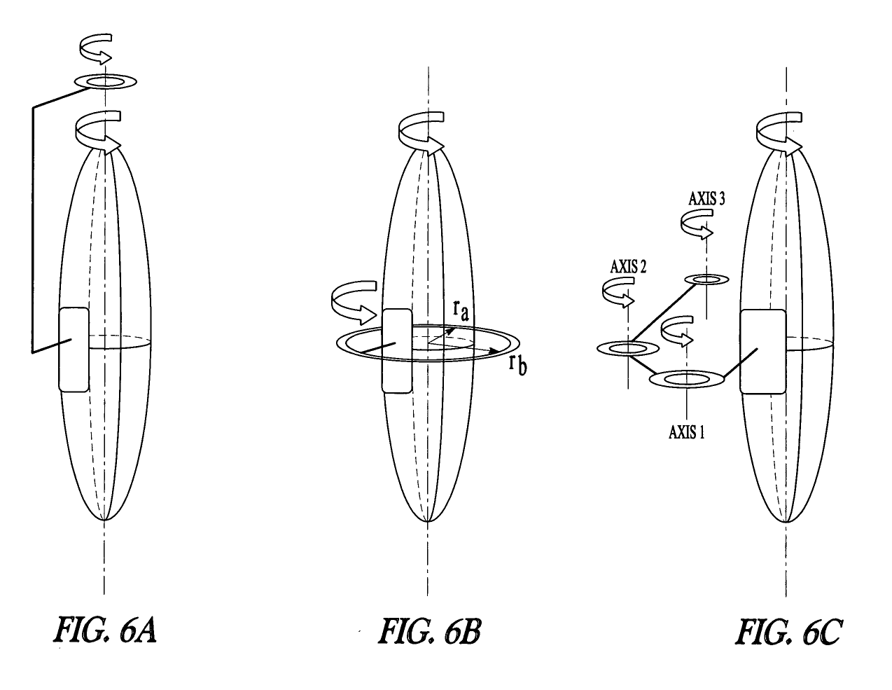

To achieve axial rotation of exoskeleton limbs, three primary exoskeletal configurations are contemplated, as illustrated in FIG. 6. FIG. 6 illustrates some exoskeleton configurations that achieve rotation about the long axis of a limb segment.

The first two configurations involve a single DOF bearing with its axis of rotation aligned collinearly with the approximate anatomical axis of rotation of the segment, while the third configuration involves a first axis that is displaced from the anatomical axis and a minimum of two additional non-collinear axes. In the first two configurations, the exoskeleton joint can be placed at either end of the long axis of the segment, as shown at FIG. 6A, or axially between the ends of the segment, as shown at FIG. 6B, using a bearing of minimum radius, r_{b}, greater than the maximum anthropometrical radius, rₐ, about the corresponding segment axis. The additional axes of the third configuration are used to correct for non-colinearity of the first axis with respect to the rotating segment.

FIG. 6A shows one configuration that allows for proximal placement of heavy components such as bearings and actuators, reducing inertial effects on power consumption, however, the placement is undesirable due to human-machine interferences during shoulder abduction. FIG. 6C illustrates a configuration the avoids the interferences by displacing the joint axis laterally from the segment axis of rotation. However, the two additional joints, adding undesired weight and complexity to the design, are used to maintain proper rotation as was achieved in previous configurations through the use of a single joint. The second configuration, shown in FIG. 6B, offers an alternative single-DOF solution where the human-machine interferences associated with the configuration of FIG. 6A can be removed. Full 360-degree bearings in this arrangement interfere with the torso when the arm is at rest or during motions that place distal arm joints near the body. Alternatively, these interferences can be removed through substitution of the full bearing with a partial bearing where the bearing track is affixed to the proximal exoskeleton link.

One example of the present subject matter uses a on-mobile platforms for immediate upper-limb exoskeleton technologies, and consequently, more user-friendly mHMI's, based on strength-to-weight considerations. Other materials and lighter and more powerful electric motors, as well as energy-to-weight ratios of power supplies can be used for a mobile platforms for partial-body upper-limb exoskeletons. In one example, a full body exoskeleton supports the existing weight of power supplies, onboard controllers, and other upper-limb hardware.

E. Singularity Placement

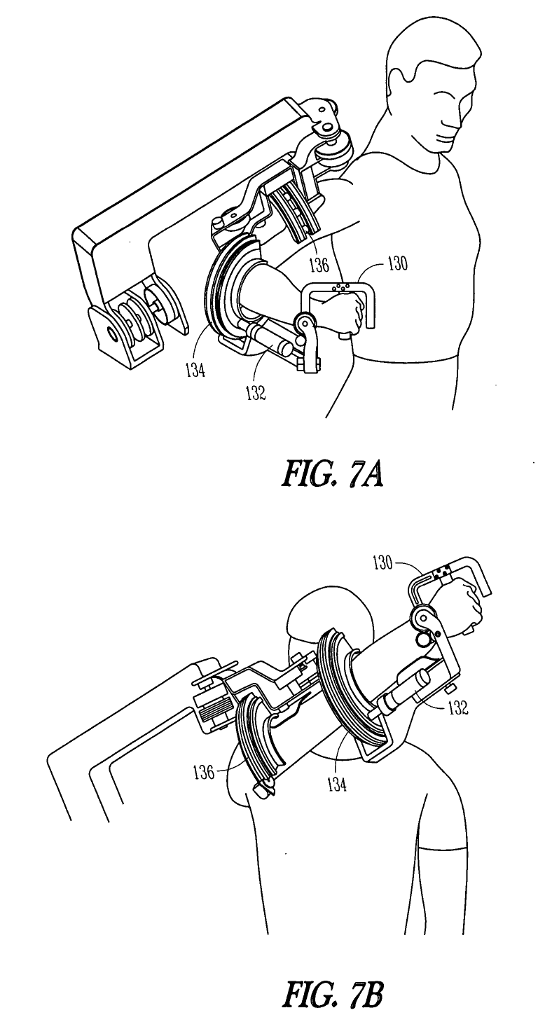

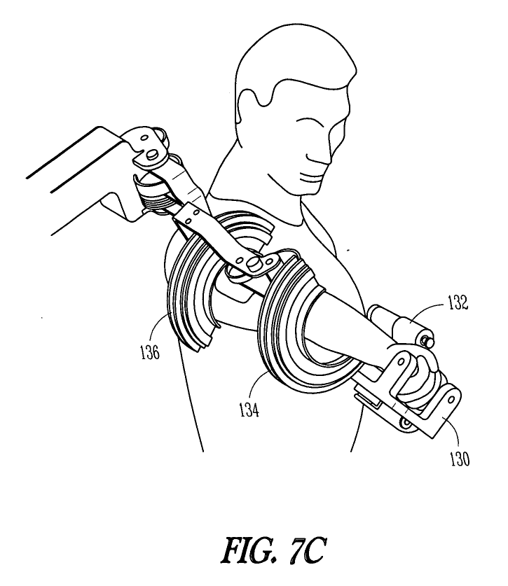

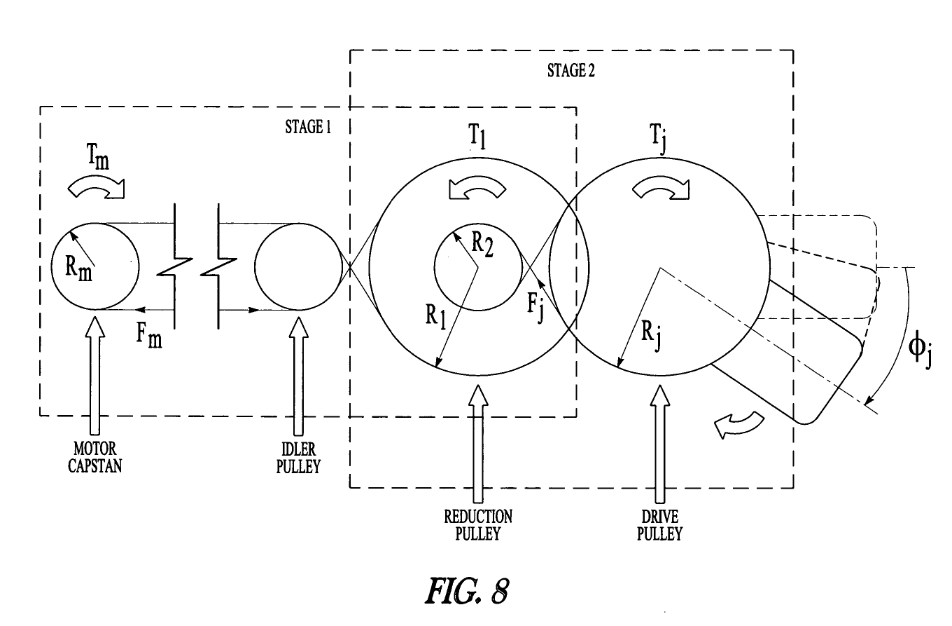

A singularity is a device configuration in which a DOF is lost or compromised as a result of alignment of two rotational axes. To mitigate complications caused by a singularity of the exoskeleton, the singularity is positioned outside or at the edge of the anthropometric reachable workspace of the human arm. For the exoskeleton arm, a singularity occurs when joints 1 and 3 are aligned, as illustrated in FIGS. 7A and 7B. Also, a singularity occurs when joints 3 and 5 are aligned, as illustrated in FIG. 7C. Then full elbow extension example is illustrated in FIG. 7C. Each of these singular configurations take place at or near the edge of the human workspace, thus leaving the majority of the workspace free of singularities. Moreover, for ease of movement in any direction, singular axes are placed orthogonal to directions where isotropy is of highest importance. For the singularity placement shown in FIG. 7, isotropy is set at 42.5 degrees of shoulder flexion and 26.4 degrees of shoulder abduction. While other values can be selected, these values lie in the median of shoulder range of motion (ROM). Some of the elements illustrated in FIG. 7 include hand piece 130, lower arm link 132, circular bearing 134 for the lower arm, circular bearing 136 for the upper arm, and upper arm link 138.

In one example, and to allow some user-specific flexibility in the design, the singular position is movable in 15-degree increments. For the placement shown in FIG. 7, the singularity can be reached through simultaneous extension and abduction of the upper arm by 47.5 and 53.6 degrees, respectively, as shown in FIG. 7A. Similarly, the same singularity can be reached through flexion and adduction by 132.5 and 53.6 degrees, respectively, as shown in FIG. 7B.

Another aspect to consider when placing singularities is mechanical isotropy. For ease of movement in any direction, singular axes should be placed orthogonal to directions where isotropy is of highest importance. For the singularity placement shown, isotropy is maximized in 42.5 degrees of shoulder flexion and 26.4 degrees of shoulder abduction—these values lie in the median of shoulder ROM.

Due to the unique placement of the shoulder singularity, pure shoulder flexion is achieved through a combination of rotations about joints 1 and 2. Additionally, this placement moves the region of highest shoulder joint isotropy into the area of the workspace most often utilized during functional tasks.

F. Power Transmission

In the present subject matter, weight is a factor that is balanced against strength or rigidity. Placement of the motors, relatively heavy components in the exoskeleton system, has an impact on the overall performance of the system and the quality of its interaction with the human operator.

The transmission of power from one location of the exoskeleton to another is achieved through a variety of means such as shafts, cables, fluid lines (for example, hydraulic or pneumatic), and gear trains. In one example, a cable-drive system is used as a balance between weight and strength or rigidity.

1. Cable Drive Systems

The cable drive transmission is able to transmit loads over long distances without the friction or backlash inherent to gears. Backlash is reduced through the structural continuity of the cable, thus enabling a direct link between the driving shaft and the shaft or link being driven. Cable-driven systems, including one stage of speed reduction for joints 5-7 and two stages of speed reduction for joints 1-4, are used to transmit torques from the actuators to the various joints. An I-beam cross section shape is used for the links, thus allowing bilateral cable routing, as well as high structural stiffness and strength.

Cable drives are also biomimmetically referred to as tendon-drives.

2. Selection and Placement of Actuators (Motors)

The drive motors are relatively heavy, and are mounted on the stationary base for joints 1-4. The remaining three drive motors, whose torque requirements (and weight) are lower, are positioned on the forearm. As each motor carries the weight and inertia of the more distally placed motors, the importance of high power-to-weight ratio increases from shoulder to wrist. Shoulder and elbow joints are each driven by a high torque, low power-to-weight motor (6.23 Nm, 2.2 Nm/kg), while wrist joints are driven by a lower torque, high power-to-weight motor (1.0 Nm, 4.2 Nm/kg). In one example, the drive motors are rare earth, brushed motors (Maxon Motor, Switzerland).

3. Reductions—Single-Stage and Two-Stage

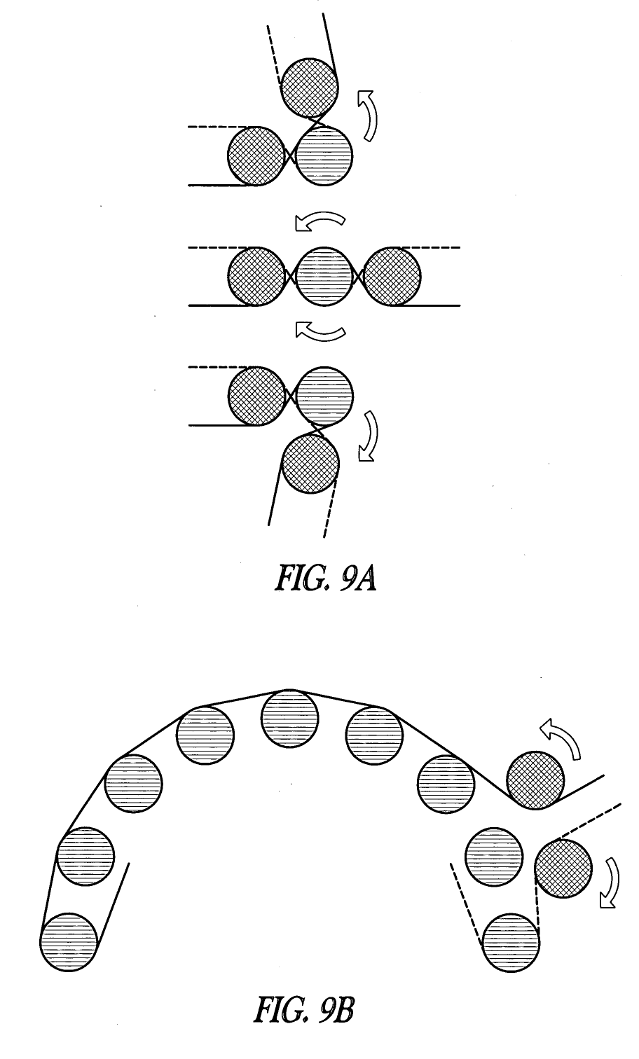

Pulley arrangements are used to create speed reductions in cable transmissions. Neglecting frictional losses, power throughout the transmission remains constant while tradeoffs between torque and angular velocity can be made. At the motor, the torque is low while angular velocity is high, whereas at the joint, torque is high and the angular velocity is low. Lower torque corresponds to lower cable tension in stage 1, as shown in FIG. 8, resulting in less strain, and therefore, less stretch per unit length of cable. FIG. 8 illustrates a two stage reduction drive. Minimizing the length of stage 2 and routing the cable in stage 1 through the majority of the robot maximizes the overall transmission stiffness. Two-stage pulley reductions have been implemented in joints 1-4, whereas the wrist includes a single-stage pulley reduction following a single-stage planetary gear reduction. In one example, total reductions for each joint are as follows: ˜10:1 (Joints 1-3), ˜15:1 (Joint 4), ˜30:1 (Joints 5-7).

4. Cable Selection, Cable Routing, and Tensioners

The joint cables are routed through or around joint axes and configured to maintain a constant length in order to achieve mechanical joint ranges of motion that match those of the human arm.

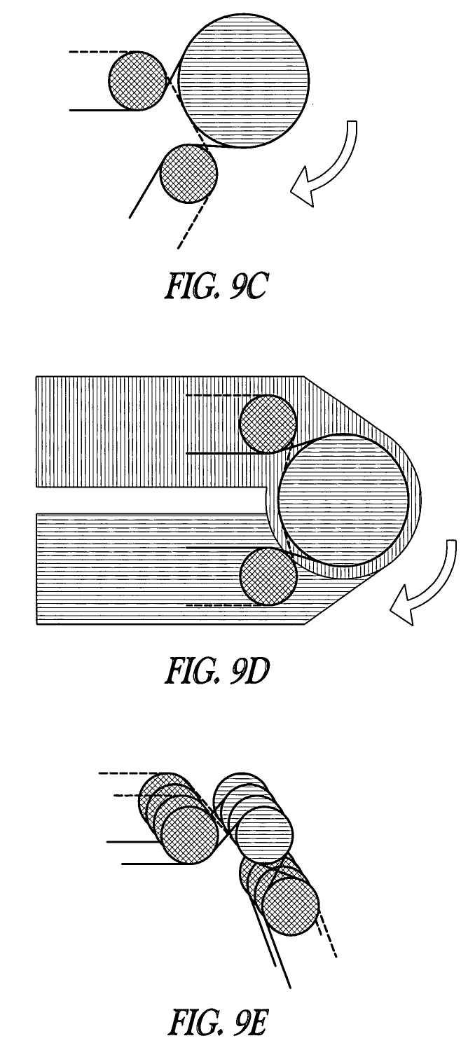

The cables are routed using various methods, including those illustrated in FIG. 9. In 90-degree and 180-degree joint configurations, the cable is wrapped around a pulley, called the joint idler pulley, which is concentric with the axis of revolution, as illustrated in FIG. 9A.

Axial joint configurations can include a series of 9 pulleys with each located at a constant radius from the axis of revolution and together acting as a single larger-diameter joint idler pulley, as illustrated in FIG. 9B.

To maintain constant cable length, the cable is to remain in contact with the joint pulley at all times. The sequence shown in FIG. 9A shows the extent ofjoint motion using three equal diameter pulleys. In the extreme positions, the shorter length of cable is tangent with the joint pulley and is therefore defined as the joint limit. FIG. 9C illustrates the effect of increasing the joint pulley radius r, on the amount of clockwise rotation before reaching the joint limit.

FIG. 9D illustrates a 90-degree joint configuration and illustrates how an increased joint pulley radius r and offset d equal to r allow links to fold to an angle of zero degrees. Each pulley actually represents a stack of two pulleys per DOF passing through the joint. Two DOF, for example, would have a stack of four pulleys, as illustrated in FIG. 9E with two pulleys representing the agonist muscle group and two pulleys for the antagonist.

In one example, high-stiffness I-beam cross-sectional members are used for the mechanical links. The I-beams also enable bilateral routing of cables and provide lightweight strength.

In one example the cable is fabricated of steel or stainless steel and is sometimes referred to as wire rope. The cable is available in a variety of strengths, constructions, and coatings. Although cable strength generally increases with diameter, the effective minimum bend radius is decreased. Cable compliance, cost, and construction stretch generally increases with strand count. A 7×19 cable, includes 133 individual strands, offers moderate strength and flexibility and is suitable for use with pulleys as small as 25 times the cable diameter. Applications requiring high strength cables and small diameter pulleys, less than 1/25^{th} the cable diameter, should utilize a higher count cable construction. One example of the exoskeleton uses both 7×19 and 7×49 cable constructions, where cable diameters were selected according to the following equations for cable stretch, s, and cable factor, CF,

$$\begin{matrix}s = ( \frac{0.0169 \cdot F}{{F}_{BS}} + 0.0005 ) \cdot L & ( 2 ) \\ CF = \frac{F}{{D}_{c} \cdot {D}_{p}} & ( 3 )\end{matrix}$$

where F is the cable tension, F_{BS} is the cable breaking strength, L is cable length and D_{c} and Dₚ refer to the outer and root diameters of the cable and pulley, respectively.

The cable tension, F, in Equations 2 and 3 can be computed at the motor in stage 1, Fₘ, or at the joint in stage 2, Fⱼ, based on the joint torque, Tⱼ, using equations 4 and 5,

$$\begin{matrix}{F}_{m} = \frac{{T}_{j}}{{N}_{2} \cdot {R}_{1}} & ( 4 ) \\ {F}_{j} = \frac{{T}_{j}}{{R}_{j}} & ( 5 )\end{matrix}$$

where N₂ is the stage 2 pulley reduction, Rₗ is radius of the larger diameter reduction pulley, and Rⱼ is the radius of the drive pulley at the joint.

Optimal cable factors for 7×19 and 7×49 cable constructions are less than 0.46 kg/mm² (650 lbs/in²) for nylon coated cables and decreases about a third using bare cable for a 2M cycle life. Note that CF is not a measure of tensile stress (psi) in the cable, but rather a measure of the accumulation of fatiguing stresses due to bending (see Eq. 3).

In one example, all cables are terminated via multiple wraps around capstans of varying diameters. Movement of exoskeleton joints are achieved by wrapping of the cables at one end of the grooved capstans while simultaneously unwrapping at the other. This motion results in a lateral motion of the cables along the length of the capstans, accompanied by slight increases and decreases in cable length. Joint motions that cause significant changes in cable length will result in one of two undesirable effects: either excessively high cable tension, reducing the life of the cables and bearings, or excessively low tension, potentially developing slack, transmission backlash, or even cable derailment. To prevent such occurrences, trans-joint pulley arrangements are kept in contact with the joint pulley at all times, and lateral deviations of the cable at all cable termination sites were limited to 2.5 degrees.

Cable tension is maintained, in various examples, by taking up slack at one or both ends of a cable or by taking up slack at a position along the length of the cable run. The slack can be taken up at a cable end by a turnbuckle device or other threaded adjuster. The slack along a length can be taken up by displacing a portion of the cable from a straight run alignment. A stationary or moving component can be used to displace the cable.

### III. CONTROL SYSTEM (HMI)

In one example, the HMI is placed at the neuromuscular junction by using surface electrodes to measure bio-signals involved in the movement of the joint. The bio-signals correspond to myosignal intentions of muscle contraction. In addition, the HMI is placed at the neuromuscular junction by simulating and predicting the functions of the human body's subsystems and organs using the interface level (myosignals) down to the lower levels of the physiological hierarchy (skeletal muscle forces and moments). The term myoprocessor is used to describe the element of the system that simulates the human skeletal muscles behavior and provides an estimation of the muscle forces.

During the electro-chemical-mechanical time delay (EMD), the system gathers information regarding the physiological muscle's neural activation level based on processed EMG signals, the joint positions, and joint angular velocities. This information is provided to the myoprocessor, which in turn predicts the moments that are going to be developed by the physiological muscles relative to each joint. The predicted moments are fed to the exoskeleton system such that by the time the physiological muscle contracts, the exoskeleton has amplified the joint moment by a pre-selected gain factor and assisted the movement. Part of the time gained by using these predicted muscle forces is employed by the electromechanical subsystems of the powered exoskeleton to compensate for their own inherent reaction time.

Controlling the exoskeleton can be implemented in a hierarchical fashion using two levels. The high-level control includes the graphical user interface (GUI) and physical human machine interfaces (HMI) allowing the patient, the therapist, and the engineer to track and control different aspects of the system operation. The low-level servo controller is the underlying component that facilities the physical interaction between the human operator and the exoskeleton system, using sensor information as inputs while generating command signals to the exoskeleton system actuators as outputs.

In one example, the high-level control includes three different graphical interfaces. The patient (operator) views a virtual environment through a HMD and physically interact with the exoskeleton arm. The GUI of the therapist includes the view of the patient as well as clinical information such as EMG signals from the muscles, descriptions of the kinematics and dynamics of the joints, joint limits, and traces of the arm positions. The engineering GUI is used mainly for development and debugging phases and provides low-level information regarding the servo control, and the ability to change the servo controller's parameters in real-time. The human-machine interface component presents the information for a therapist (for example) that is most relevant to the patient's treatment.

Impedance control is used as the low-level servo control mode. The impedance control mode unites the force sensors located in each physical interface between the human arm and the exoskeleton. The general operational concept of this control mode is that higher forces applied to the exoskeleton arm by the operator result in faster corresponding motions of the exoskeleton, and vice versa. The joints' velocities measured by differentiating the joint position are used as feedback signals. Designing and implementing the control algorithms is based on a model of the kinematics and dynamics of the exoskeleton arm and hand. System identification is performed in order to define internal parameters of the plant.

The control concepts implemented in the exoskeleton includes: (i) joint space control, and (ii) Cartesian space or end-effector control. Using the joint space control the exoskeleton operator can convert a given task into a desired path for the exoskeleton joints. The operator is able to control each joint separately, and it is up to the operator to set the right joint angles to achieve the required position/orientation of the hand. In one example, using an end-effector control, the system generates the desired joint angles automatically without user intervention, in order to achieve the position/orientation of the hand using the anthropomorphic constraints. In one example, the operator has direct control on the position/orientation of the hand only.

To establish an HMI at the neuromuscular junction. The system measures the bio-signals from the user. In one example, the myosignals of the muscles involved in the joint's movement are measured by surface electrodes, using non-invasive techniques. In addition, the system simulates and predicts the functions of the human body subsystems and organs from the interface level (myosignals) down to the lower levels of the physiological hierarchy (skeletal muscle forces and moments). The term myoprocessor is used to define the component of the system that simulates the human skeletal muscles behavior and provides an estimation of the muscle forces. The myoprocessor can be implemented based on Hill-based muscle models and Neural Network models.

By establishing the interface at the neuromuscular level, the system is able to estimate the forces that will be generated by the muscles before the muscle contraction actually occurs. This concept takes advantage of the electro-(chemical)-mechanical delay (EMD), which inherently exists in musculoskeletal system.

The exoskeleton and the human are mechanically linked through contact force sensors that generate feedback signals to correct errors of the myoprocessor prediction. This results in a well coordinated and natural movement of both the human arm and the exoskeleton system with amplification capabilities provided by the exoskeleton such that the operator feels a scaled down version of the external load. Moreover, since both the human arm and the exoskeleton share the same source of neural information, the operator feels that the exoskeleton is naturally integrated as an extension of his or her arm, providing intuitive control of the task. In persons with preserved skin sensation and joint position sense (proprioception), these sensory modalities facilitate precise control of the device.

A. Introduction

A variety of control algorithms can be used with the present subject matter, including position, force—impedance. To trigger motion in the exoskeleton, the operator either moves part of his/her upper limb or applies a force on the exoskeleton system.

For a neural control system, the neural HMI (nHMI) is set at the neuro-muscular level of the human physiological hierarchy, using processed sEMG signals as a command signal.

B. Hill Model and Neural Control

Two control concepts for operating the exoskeleton are considered (i) joint space control, (ii) Cartesian space or workspace control. Using the joint space control the exoskeleton operator converts a given task into a desired path for the exoskeleton joints. The operator is able to control each joint separately, and it is up to the operator to set the right joint angles to achieve the required position/orientation of the hand. On the other hand, in workspace control the system will generate the desired joint angles automatically without user intervention, in order to achieve the position/orientation of the hand using the anthropomorphic constraints. The operator has direct control on the position/orientation of the hand only. The control laws are incorporated in an hierarchical fashion in which low level control will compensate gravity and inertial effects and high level control interprets the operator commands like sEMG force/torque, position, velocity, and impedance to provide natural integration between the human and the exoskeleton system.

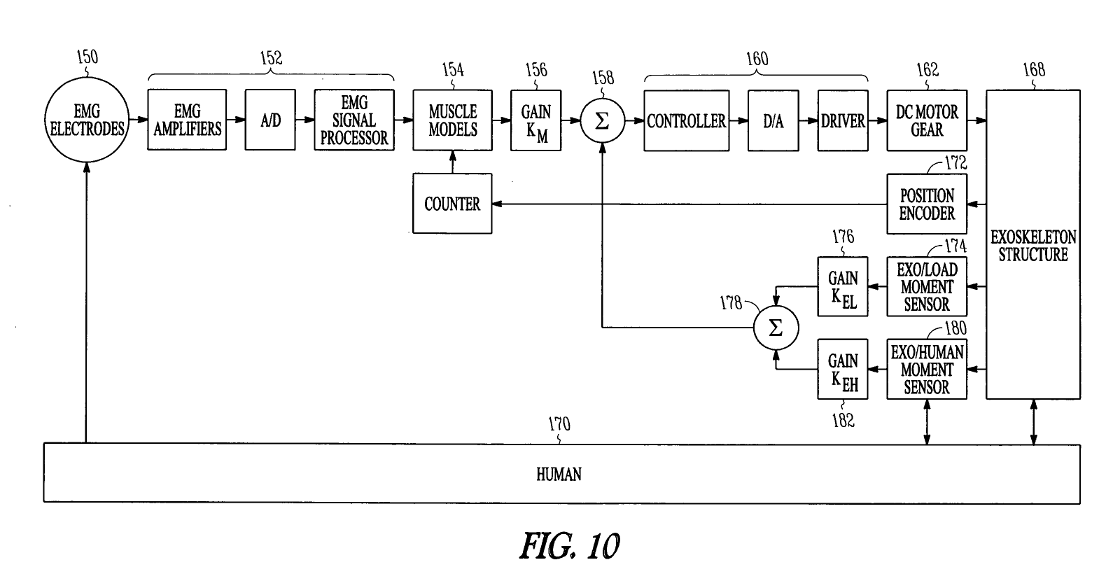

A block diagram of a one degree of freedom system is shown in FIG. 10. In this system, the single DOF is aligned with the elbow joint. The primary input to the system is the EMG signals of thejoint flexion/extension muscles group. These raw EMG signals are processed for evaluating the muscle normalized activation level. This muscle activation level, in conjunction with the joint kinematics, is next fed into the myoprocessor (muscle model) which produces an estimation of the moment to be generated by the muscles on the joint. This constitutes the primary input to the controller. The controller's inner closed loop (feedback signals) includes two sources (i) the external-load/exoskeleton load cell, measuring the effective moment applied by the load to the elbow joint, and (ii) the human-arm/exoskeleton load cell which monitors the moment applied by the operator to the system. After processing this information, the controller generates a command signal to the DC motor driver. Elements illustrated in FIG. 10 include EMG electrodes 150, signal processing 152 (including EMG amplifiers, analog-to-digital converter, and processor), muscle models 154, gain element 156, summing junction 158, signal processing 160 (including controller, digital-to-analog converter, and driver), DC motor gear 162 and exoskeleton 168. In addition, the figure illustrates position encoder 172, counter, exoskeleton/load moment sensor 174, gain element 176, summing junction 178, exoskeleton/human moment sensor 180, and gain element 182. Points of connections to human 170 are also illustrated, with arrowheads denoting the direction of information movement.

From the system perspective, the control algorithm uses three sets of feedback information: (i) dynamic feedback—the moments generated at the interfaces between the human arm, the external load and the exoskeleton structure; (ii) kinematic feedback—the joint angle measured by an encoder (including angular velocity). These signals are used by the myoprocessor; and (iii) physiological feedback—the operator uses his/her inherent biosensors and receptors (visual, muscle spindle, tendon organ, joint receptors). This physiological feedback is not implemented directly in the exoskeleton control scheme. However, it is taken into consideration by matching the exoskeleton controller frequency bandwidth to the human operator frequency bandwidth.

Two types of control are considered: (i) torque control (ii) impedance control. The control algorithm, based on a torque controller, is motivated by two concepts: (i) in operating the exoskeleton, the output of the myoprocessor (muscle model)—the reference signal, is a torque command, and (ii) for DC servo motors, the command to the linear amplifier, which in turn generates a current signal output, is directly proportional to the motor torque in the operational working range. The impedance control law uses torque commands to control angular velocity. These two control laws dictate two different modes of operation. Using torque control law entails constant application of torque in any position for compensating the gravitational force of the external load, except in the case where the exoskeleton structure is placed perpendicular to the ground. Although the load sharing is more intuitive from the operator perspective, fatigue might build up especially as a result of isometric loading conditions. However, in using impedance control the torque applied by the operator is related to the angular velocity of the joint. The exoskeleton structure maintains its position whenever the difference between the torque applied by the operator times a gain factor and the torque applied by the load is zero. This mode of operation eliminates any fatigue during isometric condition but may be less natural to operate for the user perspective.

Two types of models can be considered: (i) Hill-based model and (ii) Neural Network. According to one example having a single degree of freedom, the task used for evaluating the muscle models' performance was the flexion-extension movement of the forearm using a Cybex machine. For this task the muscle model inputs include the normalized neural activation of the four main flexor-extensor muscles of the elbow joint, the elbow joint angle and the angular velocity. Using these inputs, the muscle model (myoprocessor) output predicts the moment to be applied on the elbow during the movement.

One example of the present subject matter entails an exoskeleton incorporating a Hill-based myoprocessor as an assistive device. In this example, the mechanical gain achieved by controlling the exoskeleton with EMG signals was significantly higher (Gain=16) when compared to other modes of operation, which relied only on contact force sensors (Gain=8). This substantial gain difference is also associated with less muscle activity (3.2% for the maximal gain) and less mechanical work required of the operator. The results indicate the robustness of the control system in the presence of low neural activity. A subject with Tay-Sachs disease, disabled by severe muscle atrophy, was able to control the system using EMG signals, for example. The exoskeleton allowed this disabled person, who lacked the strength to flex his elbow against gravity, to achieve normal elbow movement and carry an external weight.

C. Myoprocessor

The following describes certain elements of the myoprocessor and a genetic algorithm (GA) for adjusting myoprocessors' internal parameters.

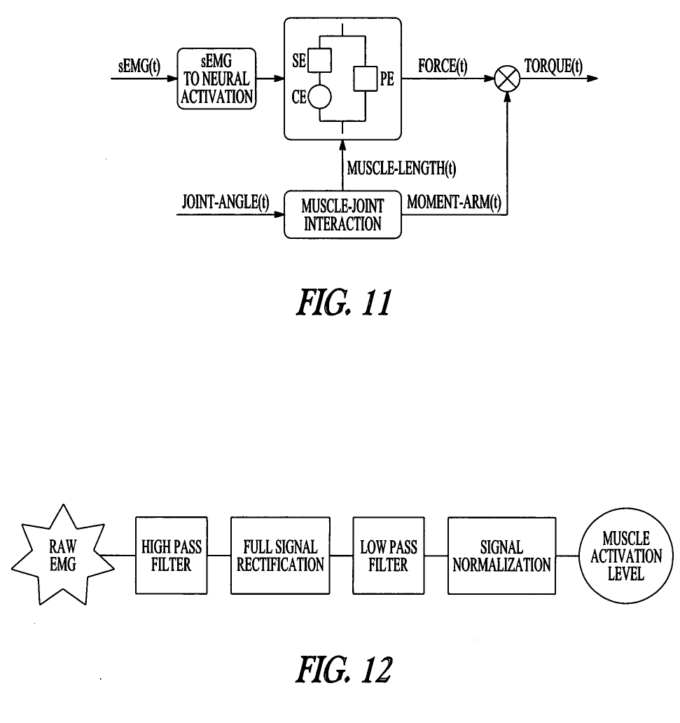

Each myoprocessor, a model of which is shown in FIG. 11, includes four modules:

1) a “neural activation module” which uses sEMG signals to estimate the degree of neural activation of the muscle;

2) a “kinematics module” which uses the joint angular positions and anatomical information to compute muscle length and moment arms;

3) a “Hill-based muscle model” which computes the force exerted by a muscle given the neural activation level and the muscle length (and lengthening/shortening velocity);

4) a “dynamics module” which evaluates muscle contribution to the joint moment as the product of muscle force and moment arm.

In one example, the myoprocessor is implemented in the Matlab/Simulink environment (MathWorks Inc.) utilizing the Real-time Workshop toolbox. Several routines have been written in C and integrated into the Simulink blocks in order to achieve real-time performance.

1) Neural Activation Module:

By using the sEMG signal as input, this module estimates the level of the neural activation (NA) for each muscle under study. The NA is a normalized signal α(t) ∈ [0,1], where α=1, where α=1 indicates a state of maximal voluntary activation and α=0 represents no muscle activation. In one example, the NA level is estimated by using the envelope of the rectified and normalized sEMG signal. The module implemented in this example includes a cascade of causal digital filters and nonlinear transformations: a) high-pass filter (cutoff frequency 20 Hz); b) notch filter (60 Hz); c) full wave rectification; d) low-pass filter (cutoff frequency 5 Hz); e) normalization with respect to the maximal isometric voluntary muscle activation levels; f) nonlinear scaling, defined by (7),

$$α ( t ) = \frac{{A}^{u ( t )} - 1}{A - 1} \qquad ( 7 )$$

where A determines the degree of nonlinearity.
In one example, all the filters are Butterworth 4th order.

2) Kinematics Module:

The kinematics module computes the length of the muscle and the moment arm for each DOF spanned by the muscle. In order to obtain these outputs, the angular positions of each joint spanned by the muscle, as well as anatomical information about the arm, are used. The muscle length and moment arms have an effect on the joint torque estimation. Several estimations of length and moment arms for the upper limb muscles can be used. Certain data is available for a selected number of muscles and they are expressed as average values or as polynomial interpolations with respect to individual joint angles. Some models account, to some extent, for the complex path of muscle from origin to insertion points. These models allow the evaluation of muscle lengths and moment arms across multiple joints, and are used in one example to represent the muscle-joints interaction. In one example, each muscle is modeled as an elastic band attached to the origin and insertion points. The muscle can wrap around virtual objects (obstacles) that simulate other anatomical structures such as muscles, soft tissues, or bones, and its path can be constrained by fixed points (called via points). The obstacles are modeled as spheres, cylinders, or combination of these two basic primitives. The muscle path is then calculated as the shortest path from origin to insertion points given the obstacle constraints. After calculating the muscle path, i.e., the muscle length, since the muscle line of action is also available, in one example, the moment arms are evaluated by using the geometrical definition (bᵢ=(\vec{rᵢ}×\hat{F})·\hat{k}ᵢ), where bᵢ is the moment arm for the joint i, \hat{k}ᵢ is the direction of the joint axis of rotation, \hat{F} is the unitary vector along the force direction and \vec{r}ᵢis the distance vector from the rotation axis to the insertion point.

One example of a kinematic module includes the following joints: glenohumeral, humero-ulnar flexion-extension, radio-ulnar pronation-supination, radio-carpal flexion-extension, and radio-carpal radial-ulnar deviation. For example the Biceps Brachii (short head) length and moment arm can be described as a function of the elbow joint (flexion/extension) and forearm (pronation/supination) angles. For analysis purposes during the validation phase, only the humero-ulnar flexion-extension, and the radio-carpal flexion-extension joints were considered active, while the others were kept in fixed positions.

3) Hill-Based Muscle Model:

This module predicts the force developed by the physiological muscle as a function of the estimated neural activity level, and of the calculated muscle's length and velocity. It is based on the phenomenological muscle model first described by Hill and refined and used by other researchers. The model includes three elements arranged on two branches. On one branch there are the passive serial element (SE) and the active contractile element (CE); on the other, there is the passive parallel element (PE), as shown in FIG. 11. Given the mechanical arrangement of the PE, SE, and CE components, the two parallel branches of the model share the same displacement (Eq. 8). In addition, the two elements in series on the same branch share the same force (Eq. 9). The total force generated by the muscle is the sum of the forces developed by each branch (Eq. 10) where F is the force and L is the length of an element.
L_{PE}=L_{CE}+L_{SE} (8)
F_{SE}=F_{CE} (9)
Fₜₒₜ=F_{CE}+F_{PE}=F_{SE}+F_{PE} (10)

Given the passive nature of the PE and SE elements, the force generated by these two elements as a function of the displacement, is expressed by the same equation with different internal parameters (Eq. 11)

$${F}_{PE , SE} = [ \frac{{F}_{max}}{{ⅇ}^{S} - 1} ] [ {ⅇ}^{( ( S / Δ {L}_{max} ) Δ L )} - 1 ] \qquad ( 11 )$$

where F_{PE,SE} is the passive force generated by the PE or the SE element, ΔL is the change in length of the element with respect to the slack length, S is a shape parameter (related to the stiffness of the element), Fₘₐₓ is the maximal force exerted by the element for the maximum change in length ΔLₘₐₓ.

The force generated by the F_{CE} element is a function of the neural activation α, of the normalized force-length function ƒₗ, of the normalized force-velocity function ƒᵥ, and of a fixed parameter F_{CE}_{ₘₐₓ} defining the maximal force the element can generate

$$\begin{matrix}{F}_{CE} = a \cdot {f}_{l} \cdot {f}_{v} \cdot {F}_{{CE}_{max}} & ( 12 ) \\ {f}_{l} = exp ( - 0.5 {( \frac{\frac{Δ {L}_{CE}}{{L}_{{CE}_{0}}} - {ϕ}_{m}}{{ϕ}_{v}} )}^{2} ) & ( 13 ) \\ {f}_{v} = \frac{0.1433}{0.1074 + exp ( - 1.3 sinh ( 2.8 \frac{{V}_{CE}}{{V}_{{CE}_{0}}} + 1.64 ) )} & ( 14 ) \\ {V}_{{CE}_{0}} = 0.5 ( a + 1 ) {V}_{{CE}_{max}} . & ( 15 )\end{matrix}$$

ƒₗ is modeled as a Gaussian function (Eq. 13) where ΔL_{CE} is the length change for the CE element and L_{CE}_{₀} is the optimal fiber length; φₘ and φᵥ are parameters affecting the mean value and variance of the Gaussian. The force-velocity equation is defined by (Eq. 14) where V_{CE} is the CE velocity and V_{CE}_{₀} is the maximal CE velocity when F_{CE}=0. V_{CE}_{₀}, as shown in (Eq. 15), can be expressed as a function of neural activation and V_{CE}_{ₘₐₓ}, i.e., V_{CE}_{₀} when the activation is maximum (α=1). Moreover, the following relations hold for some of the parameters in the previous equations
V_{CE}_{ₘₐₓ}=2·L_{CE}_{₀}+8*L_{CE}_{₀}·α (16)
F_{PE}ₘₐₓ=0.05·F_{CE}_{ₘₐₓ} (17)
Δ_{PE}_{ₘₐₓ}=Lₘₐₓ−(L_{CE}_{₀}+L_{T}_{ₛ}) (18)
F_{SE}_{ₘₐₓ}=1.3·F_{CE}_{ₘₐₓ} (19)
Δ_{SE}_{ₘₐₓ}=0.03·L_{T}_{ₛ} (20)
where α is the percentage of fast fibers in a muscle, L_{T}_{ₛ} is the tendon slack length (other symbols have been previously defined). A surface can be generated to visually represent the force-length-velocity described by the previous equations for a maximal neural activation α=1. The surface may have infinite surfaces encapsulated underneath this surface for various activation levels. Essentially any point on and under the surface is a potential operational state for the muscle.

Given the length of the muscle (which is equal to the length of the PE element, L_{PE}) and the neural activation α, there are two main ways to compute the force generated by the muscle by using (Eq. 11)-(Eq. 15). Equation (14) can be inverted to find V_{CE} as a function of F_{SE}/αƒₗF_{CE}_{ₘₐₓ}; then V_{CE} can be integrated to obtain L_{CE}. When α(t) approaches zero this method cannot be used.

Alternatively, (Eq. 8) can be transformed into a nonlinear finite difference equation (Eq. 21). This equation can be solved numerically and in real-time by using the bisection method

$${F}_{SE} ( Δ {L}_{CE} [ n ] ) = \frac{0.1433 \cdot a \cdot {f}_{l} \cdot ( Δ {L}_{CE} [ n ] ) \cdot {F}_{{CE}_{max}}}{\begin{matrix}0.1074 + exp ( - 1.3 sinh \\ ( 2.8 \frac{Δ {L}_{CE} [ n ] - Δ {L}_{CE} [ n - 1 ]}{Δ t \cdot {V}_{{CE}_{0}}} + 1.64 ) ) .\end{matrix}} \qquad ( 21 )$$

4) Dynamics Model: The net moment developed in each joint is the sum of all the moments applied by agonist and antagonist muscles (Eq. 22). The moment developed by each muscle (Tᵢ) at a certain joint is computed by

$$\begin{matrix}{τ}_{net} = {\sum }_{i} {τ}_{i} & ( 22 ) \\ {τ}_{i} = {F}_{i} \cdot {b}_{i} & ( 23 )\end{matrix}$$

where Fᵢ is the force generated by a single muscle and bⁱ is the moment arm of the muscle for that specific joint.
B. Muscle Synergy—The Inverse Problem—Force/Torque Estimation with No sEMG Inputs

In one example of the present subject matter, 12 muscle bundles are modeled, namely Brachialis (BRA), Biceps Brachii long head (BLH), Biceps Brachii short head (BSH), Brachioradialis (BRD), Triceps Brachii long head (TLgH), Triceps Brachii medial head (TmH), Triceps Brachii lateral head (TLtH), Flexor Carpi Radialis (FCR), Extensor Carpi Radialis (ECR), Flexor Carpi radialis (FCU), and Extensor Carpi Ulnaris (ECU).

In one example, however, the sEMG were recorded from 9 muscles only, due to anatomical limitations in accessing some muscles using noninvasive techniques. Several methods can be used to address this issue. In one example of the present subject matter, the following two techniques can be used.

a) The neural activity of muscle bundles close together, measured by a single pair of electrodes, has been assumed to be the same except for a scaling factor. This approach was used to model the neural activation of the biceps BSHs and BLHs;

b) The criterion of “maximum endurance of musculoskeletal function” can be used for predicting load sharing of synergistic muscle groups. Based on this criterion, muscles with a larger cross section will share higher force then muscles with small cross sections depending also on their moment arms. The predictions from this criterion are improved when the moment arms are allowed to vary with joint angular position, as in one example of the present subject matter. The “maximum endurance of musculoskeletal function” criterion can be used to model the force and torque exerted by the Brachialis muscle. An equivalent two agonist model can be defined between the BRA and the BSH, BLH, and BRD lumped together. Then, the BRA force has been computed as follows:

$$\begin{matrix}{F}_{BRA} = {( \frac{{b}_{BRA}}{{b}_{Σ}} )}^{1 / 2} {( \frac{{F}_{BRA}^{max}}{{F}_{Σ}^{max}} )}^{3 / 2} {F}_{Σ} & ( 24 ) \\ {b}_{Σ} = \frac{{τ}_{Σ}}{{F}_{Σ}} & ( 25 ) \\ {F}_{Σ} = {F}_{BRD} + {F}_{BSH} + {F}_{BLH} & ( 26 ) \\ {τ}_{Σ} = {τ}_{BRD} + {τ}_{BSH} + {τ}_{BLH} & ( 27 )\end{matrix}$$

where F represent the force developed by a muscle, τ is the torque developed by a muscle for the humero-ulnar joint, b is the moment arm of each muscle, which varies according to angular position, and F^{max} is the maximum force that a muscle can exert.
C. Myoprocessor Parameters Optimization—Genetic Algorithms (GAs)

As previously noted, each muscle model has several internal parameters. To improve performance, these internal parameters are adjusted for each user. Two main types of variability can be identified:

1) variability due to the placement of electrodes;

2) variability due to anatomical and physiological differences between subjects.

The two sources of variability can be addressed by two different parameter optimization strategies. Type II variability (Intersubject) entails running a global parameter optimization once (or each time a major change takes place), in order to find the optimal set of parameters. Type I variability can be addressed with a faster optimization targeting only parameters of the sEMG to neural activation module. This latter optimization is used each time the user wears the exoskeleton. In this section a strategy for the global parameter optimization (type II) using a GA is described.

GAs are commonly used as optimization techniques because they can deal with very large search spaces, minimizing the risk of finding solutions that are only locally optimal. In this example, GAs are used for the optimization of Hill-based muscle models.

GAs find an optimal solution by using simulated evolution processes. The optimal parameters search starts from an initial random population of “chromosomes,” each of them representing a set of parameters of the various muscle models, and, thus, a potential solution. The “survival of the fittest” criterion and “genetic operators” are used to reach a final optimal population. The degree of fitness of a certain set of parameters is evaluated by a problem-specific fitness function. In the present work the best “chromosome” is the one which minimizes the rms error between the torque estimated by the model and the torque estimated by a reference method.

The GA implementation follows a stepwise process.

1) Encode the parameters of the problem into a chromosome. Choose an alphabet (such as binary or real numbers) for the genes and choose selection, mutation, crossover, and fitness functions (genetic operators).

2) Create the initial population of chromosomes and estimate, using a fitness function, the fitness degree of every chromosome.

3) Create an intermediate population, selecting elements from the previous population, using the selection function (a function that privileges individuals with a higher degree of fitness).

4) Create new individuals using crossover and mutation and insert them into the population which becomes the new population (“children” substitute “parents” so that population size is stable).

5) If there is an individual whose fitness function is above a desired threshold or a maximum number of generations is reached, terminate the evolution process, otherwise start again from Step 3.

Various parameters in the model can be optimized. Analytical estimation of the sensitivity of the model for the different parameters is not trivial, since the equations are nonlinear and, thus, sensitivity changes with the working point. For isometric conditions, some indications on the more significant parameters and on the optimization strategy to be used (muscle specific or only agonist/antagonist specific) are available. Given the complexity of the problem, no definitive guidance is available for the other loading conditions.

In one example of the present subject matter, the chromosome has been designed with 121 “genes” (see Table I).

TABLE I
GENES INSERTED IN THE CHROMOSOME FOR EACH MUSCLE.
EACH GENE CORRESPONDS TO A PARAMETER OF THE
MYOPROCESSOR EXCEPT FOR GENES MARKED WITH
AN “*” WHERE THE GENE IS A SCALING FACTOR, I.E.,
THE OPTIMIZED PARAMETER IS OBTAINED BY MULTIPLYING
THE GENE AND THE NOMINAL PARAMETER

| Gene | Boundaries |
|---|---|
| A | [0.05, 1[ |
| *L_{CE}₀ | [0.8, 1.2] |
| *L_{T} | [0.8, 1.2] |
| *L_{CE}ₘₐₓ | [0.5, 1.5] |
| α | [0.25, 0.75] |
| *S_{PE} | [0.8, 1.2] |
| *S_{SE} | [0.8, 1.2] |
| O_{b} | [−5, 5] mm |
| G_{b} | [0, 1.2] |
| φₘ | [−0.1, 0.1] |
| φᵥ | [0.09, 0.8] |

In one example, eleven parameters were selected for each of the 11 myoprocessors out of the twelve modeled (the modeling approach used for Brachialis does not require optimization of parameters).

The following parameters were optimized in one example.

sEMG to neural activation model: nonlinear scaling factor (A)—the boundaries used for this value allowed the scaling to range from linearity to strong nonlinearity; there is no clear physiological range for this parameter;

kinematic model: moment arm gain factor (G_{b}) and offset (O_{b}); these two values define the linear transformation of the moment arm in (Eq. 28), where b is the moment arm and b̅ is the average moment arm—the boundaries used for these two parameters allowed the optimization of the moment arms but they do not have physiological meaning
\tilde{b}=(b− b̅)G_{b}+ b̅+O_{b}. (28)

Hill model: optimal fiber length (L_{CE0}), maximum force (F_{CEmax}), and tendon slack length (L_{T}_{ₛ})—the boundaries chosen for these parameters allowed their variation in the range ±20%, ±50%, and ±20%, respectively, with respect to the nominal values (see Table II); the values presented in the literature for these parameters show a significant dispersion due to the different measurement conditions (measurements on cadaver, cryo-sections, male, female, old, young, etc.); the boundaries chosen allow for optimization still maintaining physiological significance; moreover, in the optimization routine, a constraint has been introduced in order to guarantee that Lₘₐₓ>L_{CE}_{₀}+L_{T}_{ₛ}; fraction of fast fibers (α)—this parameter has been constrained to vary between 25% and 75%; shape parameters (S_{PE}, S_{SE}) of the passive elements—these parameters can be adjusted in a ±20% interval around the nominal values of Table II; however it is difficult to determine a physiological range for them; and parameters of the force-length equation—these values are allowed to vary between the intervals shown in Table I, so that the qualitative shape of the force-length function is maintained.

Some of the nominal values of parameters for each myoprocessor are listed in Table II. The nominal values not listed in the tables are:
A=1,O_{b}=0,G_{b}=1,φₘ=0.05, and φᵥ=0.19.

TABLE II
NOMINAL PARAMETERS FOR THE MYOPROCESSOR MODEL
BASED ON [24] AND [30]

| | Lₘₐₓ | L_{CE}_{₀} | L_{T} | F_{CE}ₘₐₓ | α | | |
|---|---|---|---|---|---|---|---|
| Muscle | [cm] | [cm] | [cm] | [N] | [%] | S_{PE} | S_{SE} |
| BSH | 40.46 | 13.07 | 22.98 | 461.76 | 56 | 9 | 2.8 |
| BLH | 41.94 | 15.36 | 22.93 | 392.91 | 56 | 9 | 2.8 |
| TLgH | 40.29 | 15.24 | 19.05 | 1000 | 66 | 10 | 2.3 |
| TMH | 18.95 | 4.90 | 12.19 | 1000 | 66 | 10 | 2.3 |
| TLtH | 28.22 | 6.17 | 19.64 | 1000 | 66 | 10 | 2.3 |
| BRD | 35.35 | 27.03 | 6.04 | 101.58 | 75 | 9 | 2.6 |
| BRA | 13.01 | 10.28 | 1.75 | 853.90 | 38 | 9 | 3 |
| FCR | 34.78 | 5.10 | 27.08 | 368.41 | 58 | 6 | 3 |
| FCU | 33.62 | 3.98 | 27.14 | 560.7 | 57 | 6 | 3 |
| ECRB | 34.53 | 5.59 | 26.87 | 553.21 | 44 | 8 | 3 |
| ECRL | 38.33 | 8.96 | 26.80 | 2.68.42 | 50 | 8 | 3 |
| ECU | 33.68 | 3.56 | 28.18 | 256.27 | 45 | 8 | 3 |

D. Experimental Protocol and Preliminary Data Processing

The experimental protocol designed to test an example of the myoprocessors can include the recording of movements for two joints of the upper limb: elbow and wrist. The flexion/extension movements of the elbow joint (0-145 range) was performed using an “Arm Curl” VR2 Cybex exercise machine (Cybex International, Inc). Each movement can be repeated three times with three different loads (4.54, 6.80, and 9.07 Kg) moving at three angular velocities (average values of 1.8±0.26, 1.4±0.13, 0.7±0.04 rad/s that are further referred to as fast, medium and slow). The joint angle was measured by a potentiometer (Midori America Corp., Fullerton, Calif.) located on the Cybex machine. sEMG signals were collected using Silver-Silver Chloride surface electrodes (In Vivo Metric, Healdsburg, Calif.) from 28 individual right upper-limb, chest, and back muscles. Electrodes are placed in order to achieve optimal signal detection. Maximal voluntary muscle activations are recorded during isometric contractions. The sEMG signals are amplified by using a custom system with eight Teledyne A0401 modules (Teledyne Inc., CA). Each EMG channel had a gain of 1 K, common mode rejection ratio 100 dB, a first-order high-pass filter with a cutoff frequency 0.5 Hz and a sixth-order anti-aliasing low-pass filter with a cutoff frequency of 500 Hz. The time constant introduced by these filters can be neglected, compared to the time constant introduced by software filter used in the Neural activation module that is of the order of 80 ms. The data is sampled at 1 KHz by a 14-bit analog-to-digital card (United Electronic Industries, Canton, Mass.) using the Matlab Real-time workshop toolbox (Mathworks Inc., Natick, Mass.).

The muscular torques at each joint are estimated by using a model of the Cybex machine and the human arm dynamics described by (Eq. 29)-(Eq. 31), where R is the radius of the pulley of the Cybex machine, m is the mass, θ is the angular position (θ=0 corresponds to the elbow fully extended), I is the lumped inertia of the Cybex machine and the human arm.

τ_{ƒl} is the torque computed for flexion movements and τₑₓ is the torque for extension movements
τ_{ƒl}=R[mg+m(θ\ddot{R}+2\dot{R}\dot{θ}+\ddot{θ}R)]+I\ddot{θ} (29)
τₑₓ=−R[mg−m(θ\ddot{R}+2\dot{R}\dot{θ}+\ddot{θ}R)]+I\ddot{θ} (30)
R=R(θ). (31)

This mechanical modeling of the Cybex machine provides a reference joint torque to which the myoprocessor output is compared during parameter optimization and testing. There are possible sources of uncertainty (such as approximation of geometry, inertia, etc.) that cause an estimated uncertainty for joint torques in the range of 3 to 4 Nm (about 6%-8% of the maximal peak-to-peak measured torque).

The wrist exercises involved the use of free weights (four different loads: 0.45, 1.04, 1.41, and 2.06). Each movement was repeated three times. Wrist flexion movements were performed with the elbow flexed at 110 deg and the forearm fully supinated. Wrist extension movements were performed in the same condition but with the forearm fully pronated. The wrist position was measured by an electrogoniometer fixed to the forearm and the hand. The torques were estimated by using (Eq. 31)-(Eq. 32) where mis the free weight plus the hand weight, R is the distance from the joint axis to the center of mass of the hand and weight system, θ is the joint angle (positive values for flexion and negative for extension), and I is the inertia
τ_{ƒl}=Rmg cos(θ)+I\ddot{θ} (31)
τₑₓ=−Rmg cos(θ)+I\ddot{θ}. (32)

An error analysis similar to the one performed for the elbow joint indicated that the uncertainty in the wrist reference torques is in the range of 0.05 to 0.1 Nm (about 3.5%-7% of the maximal peak-to-peak measured torque).

E. Performance Metrics

The model predictions were assessed with respect to the reference joint torques by using three criteria: maximum error (Eq. 33), root mean squared error (Eq. 34), and correlation coefficient (Eq. 35). The root mean squared error was also used as a fitness function for the GA

$$\begin{matrix}{E}_{max} = {max}_{i} \vert τ [ i ] - \tilde{τ} [ i ] \vert & ( 33 ) \\ {E}_{rms} = \sqrt{\frac{1}{N} {\sum }_{i = 1}^{N} {( τ [ i ] - \tilde{τ} [ i ] )}^{2}} & ( 34 ) \\ ρ = \frac{{C}_{τ \tilde{τ}}}{{σ}_{τ} {σ}_{\tilde{τ}}} & ( 35 )\end{matrix}$$

where τ represents the reference torque, \tilde{τ} is the torque computed by the model, and N is the number of sample points, C_{τ\tilde{τ}} is the covariance coefficient, σ_{τ} and σ_{\tilde{τ}} are the standard deviations.

An additional parameter used to assess the performance of the myoprocessors is the percentage of time (ηₛ) the absolute error is below a specific threshold value (namely, s=4,6 Nm for the elbow and s=0.4, 0.6 for the wrist)

$${η}_{s} = \frac{{\sum }_{k = 1}^{M} 1}{N} ( ∀ k \vert \vert τ [ k ] \vert \leq s ) . \qquad ( 36 )$$

III. Results

During the first phase of the experimental recordings, flexion and extension movements of the elbow were performed; in a second phase, recordings were done during flexion and extension movements of the wrist. The kinematics (oint angles), dynamics [joint torques, estimated by using (Eq. 29)-(Eq. 27)], of the neural activation levels of some muscles can be graphically depicted as a function of time.

The angular joint positions and the neural activation levels of the muscles can be used as inputs to the myoprocessors. Some of the joint torques serve as a reference to optimize the model parameters; the remaining torque estimations have been used to assess the myoprocessor predictions. More specifically, the myoprocessor parameters of the FCU, FCR, ECRB, ECRL, and ECU muscles have been optimized by using repetition #2, 1.04-Kg load, flexion, and repetition #2, 1.04-Kg load, extension movements (thus, 2 recordings have been used during optimization and 22 during testing). The myoprocessor parameters of the BRD, BLH, BSH, TmH, TLgH, and TLtH, muscles have been optimized on repetition #2, medium velocity, medium weight, flexion movement, and repetition #2, medium velocity, medium weight, extension movements (thus, 2 recordings have been used during optimization and 52 during testing).

The performances of the myoprocessors during the test phase are summarized in Table III. The values presented refer to the metrics defined in (28)-(31). The results are averaged over the entire test dataset (test data have not been used for the model optimization).

TABLE III
AVERAGED RESULTS FOR THE TEST DATA SETS (MEAN AND STANDARD
DEVIATION) BEFORE AND AFTER OPTIMIZATION FOR ELBOW FLEXION AND
EXTENSION (EF, EE) AND WRIST FLEXION AND EXTENSION (WF, WE)

| | | Eᵣₘₑ [Nm] | Eₘₐₓ [Nm] | ρ | η₄ | η₆ |
|---|---|---|---|---|---|---|
| Non optimized | ef | 8.1 ± 2.2 | 15.3 ± 3.9 | 0.8 ± 0.1 | 0.2 ± 0.2 | 0.4 ± 0.2 |
| | ee | 9.1 ± 2.3 | 15.9 ± 4.9 | 0.84 ± 0.10 | 0.24 ± 0.16 | 0.40 ± 0.18 |
| | wf | 0.40 ± 0.1 | 0.85 ± 0.15 | 0.86 ± 0.03 | 0.64 ± 0.28 | 0.85 ± 0.16 |
| | we | 1.53 ± 0.52 | 2.85 ± 0.92 | 0.70 ± 0.05 | 0.16 ± 0.10 | 0.24 ± 0.13 |
| Optimized | ef | 4.2 ± 0.97 | 11.0 ± 3.0 | 0.87 ± 0.05 | 0.67 ± 0.11 | 0.85 ± 0.09 |
| | ee | 3.4 ± 1.3 | 9.6 ± 4.1 | 0.89 ± 0.08 | 0.79 ± 0.15 | 0.91 ± 0.09 |
| | wf | 0.26 ± 0.17 | 0.64 ± 0.24 | 0.80 ± 0.05 | 0.83 ± 0.26 | 0.92 ± 0.14 |
| | we | 0.39 ± 0.16 | 0.75 ± 0.25 | 0.42 ± 0.46 | 0.63 ± 0.28 | 0.82 ± 0.21 |

Joint torques predicted by the myoprocessors after parameter optimization can be plotted. Each plot, for example, can include three torques: 1) the myoprocessor predictions with nominal model parameters (nonoptimized); 2) the reference torque as computed by using (23)-(27); 3) the myoprocessor predictions with optimized parameters.

A notable characteristic of the myoprocessors is their ability to work in real-time. Given a specific computational power, there is a balance between the complexity and number of the myoprocessors and the capability of the hardware system to perform in real-time. The task execution time (TET) of the myoprocessors system as a function of the number of muscles modeled can be visually presented. The TET was estimated simulating a flexion movement of the elbow, with angular position described by a saw-tooth spanning the 0-145 range of motion; other joints are held in a neutral position; neural input was held constant at an activation level of 0.5 (50% of the maximal voluntary activation level). The saw-tooth had a period of 1 second. Max, min, and averages values are measured in 30-s time slots.

In one example, the hardware platform includes a PC104 with an Intel Pentium4 operating at 2.4 GHz processor and 512 Mb RAM. Nonlinearity of the TET as a function of muscle number can be observed, as a results of the different complexity of myoprocessors modeling different muscle.

### IV. DISCUSSION

This document presents the development, optimization, and integration of real-time myoprocessors as a HMI for an upper limb powered exoskeleton. As one element of a neural controlled exoskeleton, the myoprocessors provide robust, accurate joint torque predictions over a broad range of loading and motion conditions.

Both black-box and white-box approaches can be used for muscle modeling. One example used an approach in which most of the internal parameters of the myoprocessor are directly related to physiological muscle parameters. More specifically, one example of the myoprocessor includes a Hill-based muscle model together with a three-dimensional anatomical representation of the upper limb and a nonlinear sEMG-to-Activation signal processor. In addition, GAs are used to optimize the myoprocessor's internal parameters, for each specific subject wearing the exoskeleton, without the need for a priori exact knowledge of each muscle parameter. The optimization is constrained in order to prevent parameters from exceeding physiological ranges.

By some measures, the resulting model has more characteristics in common with white-box models than with black-box models (e.g., neural networks), even if the adherence to physiology of the model can be improved at several levels: some elements, such as muscle pennation, can be included in the model structure; the optimization boundaries for each parameter can be different for each muscle in order to exploit all the knowledge available for the different muscles; the Hill model and the kinematic (skeletal) model can be optimized in an intertwined way that, for example, a change in the origin or insertion point of a muscle, will be reflected in a corresponding change of tendon slack length and optimal fiber length.

As described herein, the parameter optimization has been carried out by using only a small dataset (4 recordings out of a total of 78 recordings). As indicated by the results, the ability of the myoprocessors to accurately predict the joint moment increased significantly with an optimized set of internal parameters. While optimization on a large set of data can yield better results during testing, it may not be feasible to optimize the model on all the possible upper limb movements. Note that even with a relatively small database used for the optimization process, acceptable overall performance is achievable. A small optimization database yields a model able to perform reasonably well in a broad range of conditions.

For elbow movements, the results (see Table III) indicate that the integration of myoprocessors into a single neuromuscular model of the arm is capable of predicting the joint's torque with an average Eᵣₘₛ of about 8.6 Nm when parameters are not optimized. After optimization this prediction is improved to an average Eᵣₘₛ of 3.8 Nm. Moreover, after optimization, the percentage of time the absolute error stays below 4 Nm (η₄) is increased from an average 22% to an average 73%. Also for the wrist movements the Eᵣₘₛ is more than halved after optimization and η₄ shows an increase from 40% to 73%. The predictions for the elbow joint movements showed better correlation (ρ) with the reference torques compared to the wrist joint. In particular wrist extension movements presented on average a lower ρ after the optimization, even when all the other error measures consistently improved. An explanation for this phenomenon can be provided by considering that finger flexors and extensors significantly contribute to the wrist flexion-extension torque but these muscles were not included in the model. In the case of the elbow joint, all the relevant muscles for the flexion-extension movement were included, which may explain the better ρ.

Given the synergistic behavior of the physiological muscles and the fact that some muscle[s] were not accessible using noninvasive technique[s], the “maximum endurance of musculoskeletal function” criterion has been used for predicting the contribution of the BRA muscle. This technique can be extended beyond its current use to allow further reduction in the number of sEMG electrodes required for a satisfactory torque prediction.

Different myoprocessors are able to model muscles attached to the skeleton in different ways. Modeling more complicated cases in which the muscle wraps around several anatomical structures (multiple obstacles) requires more computational power than simpler conditions (single obstacle). By accounting for these constraints, myoprocessor complexity can be shaped to match the computational power available. One example allows the 12 myoprocessors to run simultaneously in real time with a maximum TET below 400 μs. One example can include approximately 20 myoprocessors modeling muscles of wrist, elbow, and shoulder joints and able to meet the real-time requirement of the exoskeleton main control loop (computational interval of 1000 μs).

The myoprocessor described herein provides a good balance between complexity and performance. Along with GAs for the optimization of the internal parameters for a specific user, an ensemble of myoprocessors can be used for an HMI that operates in real-time conditions.

The myoprocessor is a muscle model that performs real time processing of input signals, including the muscle activation level and joint kinematics, in order to predict the muscle force or the moments generated by a synergetic group of muscles e.g. flexor or extensor. The muscle activation level is defined as the percentage of the neural activity of the muscle during maximal isometric voluntary contraction. The algorithm for evaluating the normalized muscle activation level (FIG. 12) can be used in the field of biomechanics and it is digitally implemented into the real-time control system. The algorithm includes: (i) a high-pass filter for filtering low frequency artifacts associated with the fact that the muscles are moving during their contraction; (ii) a full wave rectifier; (iii) a low-pass filter for calculating the signal's envelope and; (iv) signal normalization mapping the signal into the <0-1> range.

The myoprocessor processes the muscle's neural activation levels along with the joint kinematics to predict the muscle force (or moment with respect to a specific joint). This prediction is used by the exoskeleton system to generate the appropriate joint torque to assist the operator.

In one example, the I/O signals are used to identify the internal parameters of the both the Hill model (HM) and the artificial neural network (ANN). In terms of the HM both the force velocity (F-V) and the force length (F-L) function for various muscle activation can be identified. In addition to the HM, using the same I/O signals, a two layer ANN can be trained based on the data from, for example, 5 subjects.

### IV. VARIOUS APPLICATIONS

The exoskeleton is an external structural mechanism with joints and links corresponding to those of the human body. Worn by the human, the exoskeleton transmits torques from proximally located actuators through rigid exoskeletal links to the human joints. The control algorithm used to operate the device can be configured to implement different modes of operations, including, for example, the following four: (1) a therapeutic and diagnostics device for physiotherapy, (2) an assistive (orthotic) device for human power amplifications, (3) a haptic device in virtual reality simulation, and (4) a master device for teleoperation.

The exoskeleton of the present subject matter can be controlled by a stroke patient, for example, while performing task-oriented occupational therapy activities in a virtual reality (VR) environment.

In one example, the present subject matter includes hand exoskeletons, each having 9-DOF which enable dexterous and power grasping.

According to one example, virtual reality (VR), or virtual environment (VE) technology provides an immersed experience typically involving audio and visual feedback perception for the user. Robotic devices can apply forces to a user through a mechanical interface and can therefore add the sense of touch (haptics) to the experience. The combination of audio-visual and force feedback enables the creation of detail rich, engaging virtual environments.

In one example, a computer operable program is configured for establishing and managing a virtual coupling between a haptic-configured exoskeleton device and a virtual environment or virtual reality. One example includes a virtual representation of a human body along with two fully functional virtual human arms that are linked to the motion to the exoskeleton. The exoskeleton and the virtual reality are linked such that any motion generated by a person wearing the exoskeleton is presented in the virtual environment. The view is reflected to a pair of virtual reality goggles or eyeglasses worn by the user. In addition, joint velocities and joint toques are represented as vectors that are linked to each joint of the arm. Any gain factor can be introduced between the actual joint angle of the arm and their virtual representation in order to enhance arm movements.

One example of the present subject matter can be used for rehabilitation treatment for hemiparetic upper limb of patients with chronic stroke. The treatment utilizes an exoskeleton incorporated into an immersive virtual environment that facilitates three-dimensional arm movements. The combined effect of gravity compensation as a perturbation field can be gradually introduced by the exoskeleton along with an active assistance implemented as an impedance control. A treatment strategy which involves a gradual increase of a gravitational field from a micro-gravity field (weightless motion—0G) to a full gravity field (1G) along with a gradual decrease in robotic assistance can improve the motor recovery process. The combination of gradual increases in gravitational effects with decreased assistance, introduced in a cyclic pattern over multiple treatment sessions, may result not only in improved motor recovery, but to an extent greater than that which is achievable through full (0G) gravity compensation alone. There may also be improved ability to perform self-care activities using the hemiparetic limb.

An example of the present subject matter can also provided a robot-assisted quantitative measurements of upper limb impairment. Dynamic motor tests that employ virtual reality can be conducted using patients with chronic stroke, along with a number of conventional (non-robot-assisted) measures of impairment and disability.

The Virtual Reality Environment

Virtual environment may enable better control and provide more flexibility in creating the environment with which the patient can interact, as compared to the real physical environment. This environment allows the patient to view and physically interact with virtually any object with any physical properties. In addition, it enables altering of gravitational fields as well as guiding of visual information as the user interacts with the virtual objects.

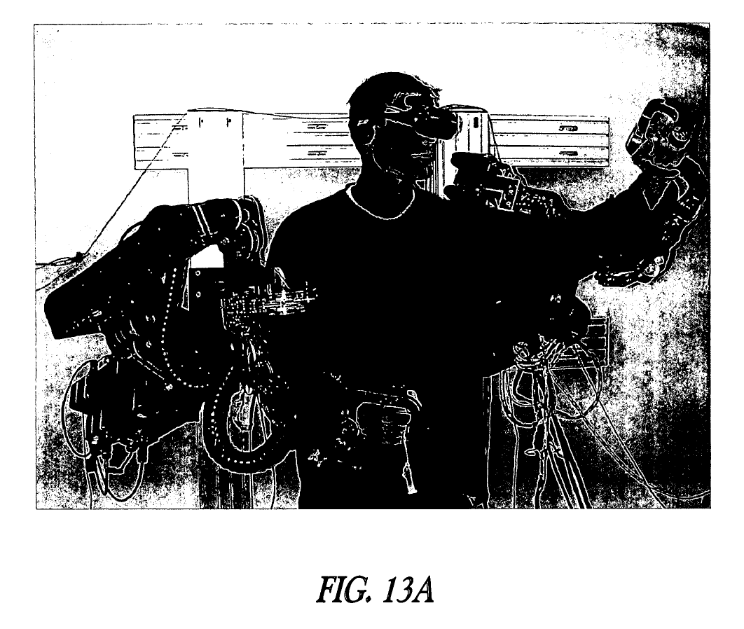

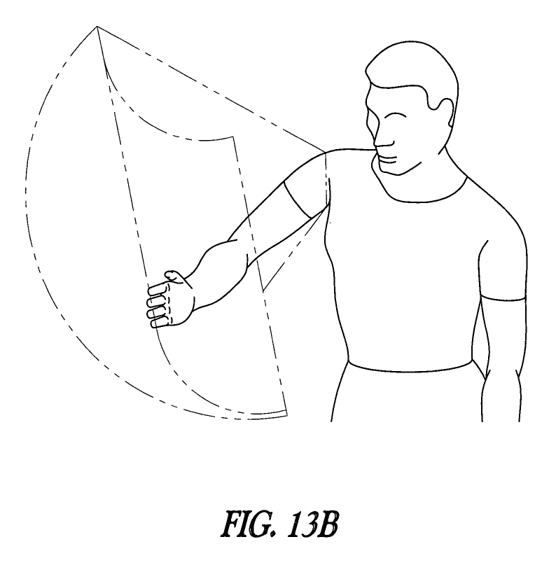

In one example, the user dons a set of head mounted displays (HMD). The HMD includes two separate screens (one for each eye) allowing for rendering of virtual reality scenes separately for each eye in order to provide 3D immersed environments, as in FIG. 13A. A head-tracking sensor is incorporated into the HMD allowing the user to view the scene from different angles. As the user moves his head, the virtual scene is rendered accordingly providing a sense of immersed environment. FIG. 13B illustrates a user with a limited range of motion of the arm. The gray volume represents a morphing of the range of motion based on the individual range of motion of each joint. A view corresponding to that of FIG. 13B is presented to the user via the HMD. In one example, the scene is visually rendered and also haptically rendered, meaning that force feedback is provided to the user once he or she touches the virtual objects or follows a path with virtual constraints. All the physical properties of a virtual object such as weight, stiffness, viscosity, texture, etc. are generated by the exoskeleton and conveyed to the user through this device. The user will feel a force as he/she presses the virtual cylinders. Virtual reality as a graphical interface can be implemented using a software package, in a OpenGL, for example. Various virtual objects, movement paths, and gravitational force fields can be implemented into the system.

As discussed elsewhere in this document, various applications can be met with one or more implementations of the present subject matter.

A. Physiotherapy—a user wearing the exoskeleton can perform task-based occupational therapy or physical therapy in an active or passive mode.

B. Assistive Device (human amplifier)—a user wearing the exoskeleton can manipulate or interact with an object or the environment in which the actual load is shared between the exoskeleton and the user.

C. Haptic device—a user wearing the exoskeleton can physically interact with a virtual reality object or scene while the forces generated through this interaction are fed back to the user through the exoskeleton conveying the shape, stiffness, texture or other physical characteristics of the virtual object or scene.

D. Master Device—a user wearing the exoskeleton can control a second robot in a master-slave relationship. As such, the user (master) can control a remote (slave) robotic system in a teleoperation mode, where the exoskeleton reflects back to the user the forces generated as the slave robot interacts with the environment. In one example, more than one slave device is controlled by a master device.

### V. OTHER EXAMPLES

In one example, the range of motion for each joint at the shoulder, elbow and wrist is approximately 180 degrees, however ranges above or below this value are also contemplated.

In one example, the exoskeleton uses internal-external rotation joints and pronosupination joints that fully enclose the arm. In this case, the user inserts their arm from one end and slides it axially down the length of the arm.

In one example, the exoskeleton includes open mHMI's for both upper and lower arm segments, thus allowing the user to don the structure more comfortably.

As a result of the placement of the shoulder singularity, as described elsewhere in this document, pure shoulder flexion is achieved through a combination of rotations about joints 1 and 2. In addition, this placement moves the region of highest shoulder joint isotropy into the area of the workspace most often utilized during functional tasks.

In one example, the axes at the wrist are anthropometrically correct in that there is a slight offset between the flexion-extension and radial-ulnar deviation axes. In one example, this offset is omitted.

One example includes a spring or other energy storage device to provide the drive power to the actuators.

One example uses a brake mechanism to control the position or velocity of a joint.

The present subject matter includes the exoskeleton as well as the control environment for a skeleton.

In one example, the system is for use in a surgical or medical environment wherein a user in a remote location is able to control a robot or other structure to deliver medical care. This may include manipulating objects, performing surgery, examinations, or providing therapy. In one example, haptic information is provided to the user. In one example, a minimally invasive surgical procedure can be performed using the present subject matter.

One example of the present subject matter is configured to be worn by a user. The position and alignment of the rotation axis of the exoskeleton joints relative to the user's anatomical joints allows the user to safely manipulate the exoskeleton links without endangering the user. In one example, the user is in a seated position and the exoskeleton is mounted on a wall behind the user. In one example, the exoskeleton is mounted on a wheelchair in which the user sits.

In one example, the present subject matter is implemented as a prosthesis in that it substitutes or replaces a limb of the user. As such, the control signals for the exoskeleton are derived from other sources, such as sEMG sensors attached to other portions of the user's body, or from a master device or controller.

In one example, the present subject matter includes three physical points of contact with the exoskeleton. For example, the exoskeleton is in contact with the human arm at the hand, the upper arm and the lower arm. At each point of contact, the system exchanges energy and information between the user and the present subject matter. The exchanged information can take the form of control signals such as force or pressure. The exchanged energy can take the form of a force applied by the exoskeleton to the user or a force applied by the user to the exoskeleton. If, for example, the exoskeleton exerts a force, then the arm will be forced to move along a certain trajectory. In addition, if the user exerts a force at a point of contact, then, as a function of the control algorithm being executed, the exoskeleton will respond and move along a particular trajectory.

Gravity (or other load force) can act on the exoskeleton to exert a force on the human operator.

In addition to the examples noted herein, the system can be configured to operate in a mode between a passive state (where the user's arm is moved based on a signal received from the exoskeleton) and an active state (where the user provides the signal to move the exoskeleton). In other words, the system can be operated at a middle position along the spectrum between active and passive. In such a case, the exoskeleton provides some level of assistance and some level of resistance. This mode allows graduated assistance and resistance and may prove beneficial in a rehabilitative or training application.

The exchange of information can be at different levels. For example, the sensors can be located at the interface between the user's arm and the exoskeleton. Such sensors can be used to discern the user's intention and when a force is exerted thereon by the user, the exoskeleton responds. At another lever, the interfaces are separated. As such, a physical exchange enables exchange of energy between the exoskeleton and the user and a separate interface is provided to exchange information at a different point in the system (for example, at the neural level). If the exoskeleton does not respond in the manner intended by the user, then the user is given an opportunity to correct in which case the feedback is derived from monitoring the joint (pivot). In one example, the feedback is at the physical level and the command is disposed at either the physical level or at a different place (such as the neural level).

The point of contact for the sensors can be at various points along the length of the link and need not be located at ajoint center. For example, a strain gages can be located between the point of contact with the arm and the structure of the exoskeleton such that forces passing through the body and the system are also passing through the single point for each of the three links (upper, lower and hand).

The points of contact can be in the form of two semicircular braces, each wrapping the forearm and the upper arm, and at a handle held the user's hand. Force-torque sensors (for example, strain gages) can be located between the handle and exoskeleton and at the two braces. The sensors provide information as to load or energy.

Various types of transducers (sensors) are contemplated. For example, an image based system of transducers can be used in which landmarks (on the user or the exoskeleton) are monitored by an optical sensor. In other examples, an encoder or stepping motor is used to provide information as to position or force.

In one example, the length of an exoskeleton link is adjustable. In one example, the position and alignment of a joint of the exoskeleton is adjustable.

In one example, a myoprocessor provides control of the exoskeleton structure and the myoprocessor is coupled to a sensor that is coupled to the user's brain (either invasively or non-invasively). The brain can be trained to provide a control signal for operating the exoskeleton.

The present system can be used in a variety of applications. For example, the system can be used for physiotherapy and rehabilitation, a neural signaling device, a training device, and a haptic or virtual reality device.

For physiotherapy and rehabilitation, the present system can be used as an automatic physiotherapy device, for assessment of a disability, and as a power device.

Consider use of the present system for physical therapy or rehabilitation. When rehabilitating a patient, the therapy work transitions through various phases. Initially the work is directed to increasing the range of motion of a joint or limb. At a later stage, and after having achieved a particular range of motion, the work is directed to assisting the patient in an active manner (not just passively). At this stage, the patient is expected to provide a portion of the energy to cause the motion and the system provides some level of energy. At yet another stage of the therapy, the work is directed to strengthening the muscle capabilities and includes applying a resistive force to resist the motion of the user. At this stage, the system operates like an exercise machine.

The present subject matter can be used for the various stages of therapy. In particular, the system can be used to drive the patient limbs in order to expand the range of motion. In addition, the system can be configured to provide a full spectrum of assistance or resistance.

In the context of neural signaling, the system can be used as a power orthotic device.

For training purposes, the present subject matter can be used for simulating human performance involving, for example, manipulation of an object, or executing an artistic or athletic performance.

In addition the present subject matter allow a user to experience a haptic or virtual reality experience.

As a human amplifier, the present system allows a user to interacting in an environment with a physical object. The user can apply a force on the object and together, the user and the exoskeleton share the load. The user will feel a fraction of the load. In one example, the system can be configured so that the exoskeleton carries a selectable portion of the load.

In addition the present subject matter allow a user to experience a haptic or virtual reality experience. For example, a user can be placed in a scene of an environment that includes a virtual object rendered in three dimensions. In the virtual scene, the user can, for example, move their hand out and touch the object with a sensing element. The present subject matter allows the user to feel the object. In addition, the user can feel a force corresponding to the feedback that would be encountered upon touching the object. For example, if the user attempts to penetrate the object the feedback force may prevent the user from going inside and instead, the object may force the user's finger around to the side. Alternatively, if the object is flexible, then the user will see the deflection on a display and the user will feel the various levels of resistance corresponding to the device flexibility. The feedback information is delivered to the user using various elements of the exoskeleton, thus corresponding to the actual feedback experienced in a real environment.

In addition, the user can interact in a virtual environment having objects that affect the performance in a manner that corresponds to a real environment. For example, if a user inserts an arm in a hole, such as a tube, then in a simulated environment, then their range of motion will be affected. When inserted a short distance, for example, the range of motion of the forearm only is restricted. If inserted farther, then both the upper and lower arms are limited.

The individual joints of the present subject matter are separately controllable and can be operated individually or simultaneously. In addition, the software controls the position and movement of the various elements of the exoskeleton in a manner corresponding to the virtual environment.

Some examples of the present subject matter include a communication system, such as a communication network or a dedicated communication line that allows portion of the system to be distributed in more than one location. For example, using a communication network such as the internet, a user wearing an exoskeleton is able to participate in a haptic experience. As such, a user in Minnesota can touch and manipulate an object located in an environment existing in Washington. In addition, the user can touch and manipulate a simulate object that exists only in a virtual environment.

In another example, the user wearing an exoskeleton is able to work with and interact with a physical therapist located in a remote location. A remote therapist can hold and manipulate a patient's arm from a remote location and a haptic feedback signal returned to the exoskeleton can allow the patient to experience the resulting forces applied by the therapist.

Other examples are also contemplated. For example, an instructor fitted with an exoskeleton structure as described herein can provide interactive training to a number of students wearing an exoskeleton. In one example, a user wears both a left and right limb exoskeleton structure and one side is used to train or mirror behavior of the other side. Such an application allows a user to self-train following an asymmetric impairment or other condition such as stroke.

Conclusion

The 7-DOF exoskeleton is a relatively lightweight, high-performance system that facilitates full-workspace and ROM. Proximal placement of motors, distal placement of pulley reductions, and open mechanical human-machine-interfaces were incorporated into the design of the Exoskeleton. Additional characteristics include low inertias, high-stifffiess links, and back-drivable transmissions without backlash.

The myoprocessor enables a neural interface between the human operator and the exoskeleton system. This neural interface contributes to a natural and stable integration between the wearable robot and the user such that the user views the exoskeleton as an intuitive extension of his/her body.

The powered exoskeleton system can increase the load carrying capacity of both healthy and physically impaired operators. In the former case, it can be used as an upper limb orthosis. For a patient with a neurologic disability to employ any powered exoskeleton, he or she may have some minimal motor control abilities in order to generate EMG signals. The powered exoskeleton allows additional functional activities to be performed by patients with weakness. In persons with quadriplegia, voluntary sEMG signals are frequently obtainable from muscles with nearly complete paralysis, and the ability to generate sEMG activity can be enhanced through a short biofeedback training protocol. Measurable sEMG signals from muscles without detectable voluntary contraction force can be used with the present subject matter. Additionally, since functional use of a partially paralyzed limb improves motor recovery in many central nervous system disorders, use of the device holds the potential for enhanced motor recovery.

The present subject matter serves as an assistive device and can be worn by the user as an orthotic device. The device functions as a force feed-forward human amplifier. The joints and links of the structure correspond to those of the human body and its actuators share a portion of the external load with the operator. The human-machine interface (HMI) is set at the neuromuscular level of the human physicolgical hierarchy using the body's own neural command signals as one of the command signals of the exoskeleton. These signals are in the form of processed surface electromyography (sEMG) signals and detected by surface electrodes placed on the operator's skin. This configuration takes advantage of the electro-chemical-mechanical delay which inherently exists in the musculoskeletal system (between the time when the neural system activates the muscular system and the time when the muscles generate moments around the joints).

The myoprocessor includes a model of the human muscle running in real-time and in parallel to the physiological muscle. During the electro-chemical-mechanical time delay, the system gathers information regarding the physiological muscle's neural activation level based on processed sEMG signals, the join position, and angular velocity, and predicts, using the myoprocessor, the force to be generated by the muscle before physiological contraction occurs. By the time the human muscle contracts, the exoskeleton moves with the human in a synergistic fashion, allowing natural control of the exoskeleton as an extension of the operator's body. Rather than feeding back information to the user based on whatever information the user may be manipulating, the exoskeleton feeds force forward to move the device based on the myoprocessor.

The force applied to the user of the device represents feedback from a real or virtual scene. The force applied to the user serves to assist their movement or constrain their motion as directed by a physical therapy regimen or by elements in a real or virtual environment. Forces are applied to change or inform a user of information based on a real or virtual environment.

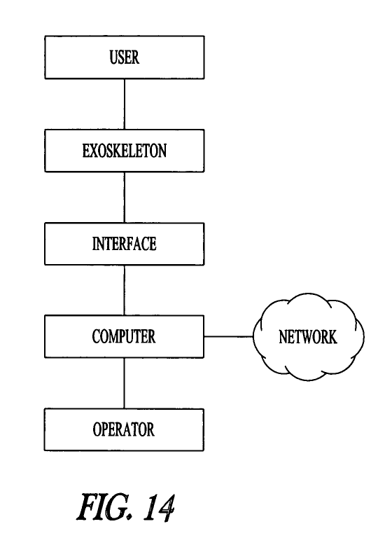

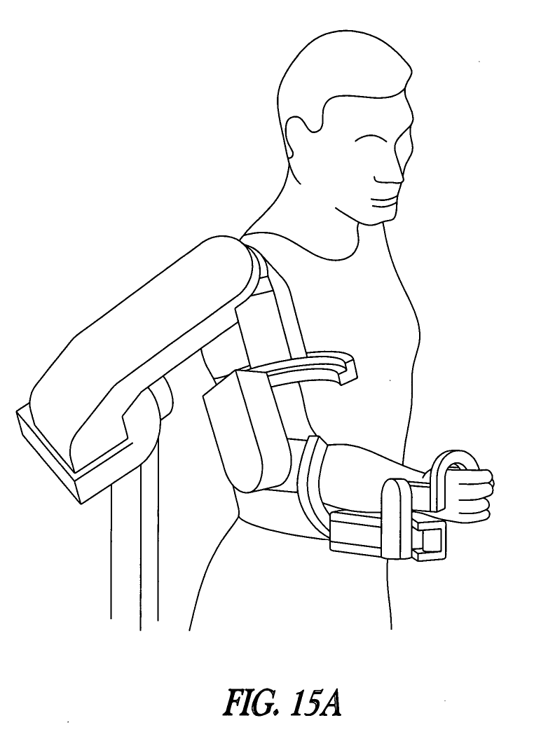

FIG. 14 illustrates a block diagram of a system according to one example. In the figure, the user is fitted with a wearable exoskeleton as described in this document. The exoskeleton includes sensors (or transducers) and number of powered limbs. The exoskeleton is coupled to an interface that receives sensor data corresponding to the joints on the limbs as well as sensor data from the user. The interface also provides driving signals to the actuators of the exoskeleton. The computer is coupled to the interface and provides a virtual reality environment, for example, or is controlled by an operator that implements a therapy regimen, for example. The computer is shown coupled to a network which allows communication with other systems. For example, a second user in a remote location can serve as a master or a slave and operate in conjunction with the user illustrated in the figure.

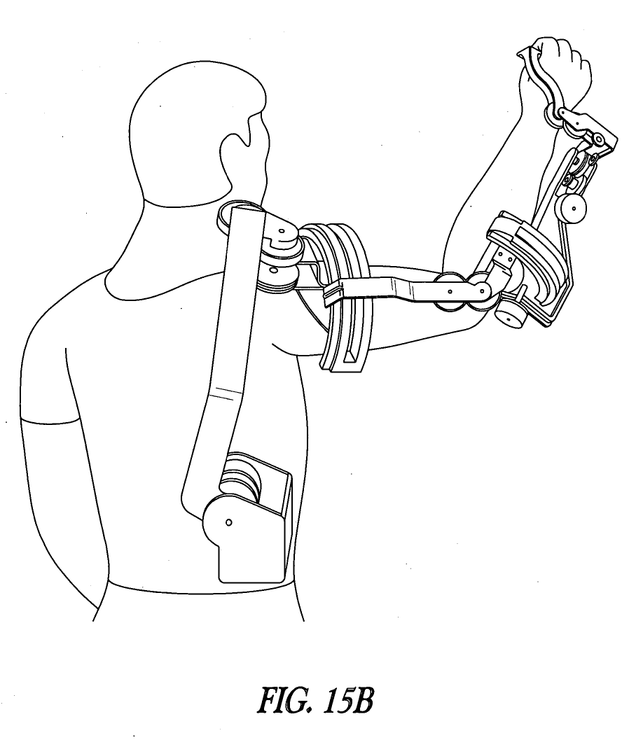

FIGS. 15A and 15B illustrate perspective views of a model human wearing an exoskeleton of the present subject matter.

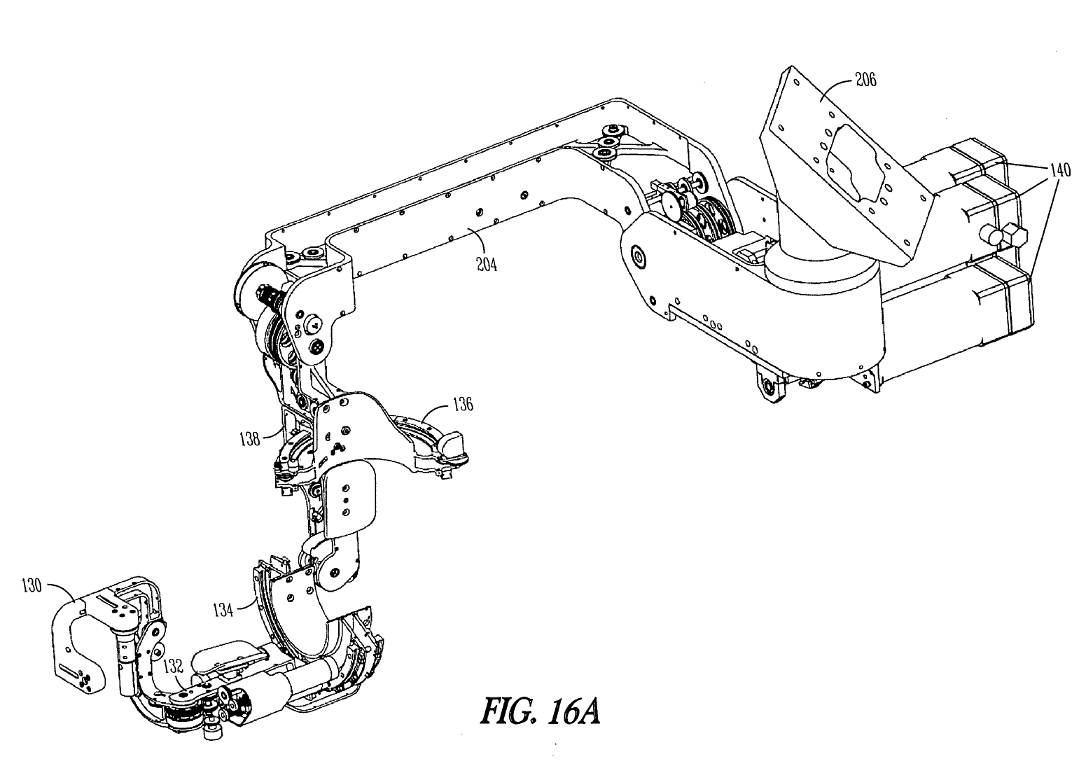

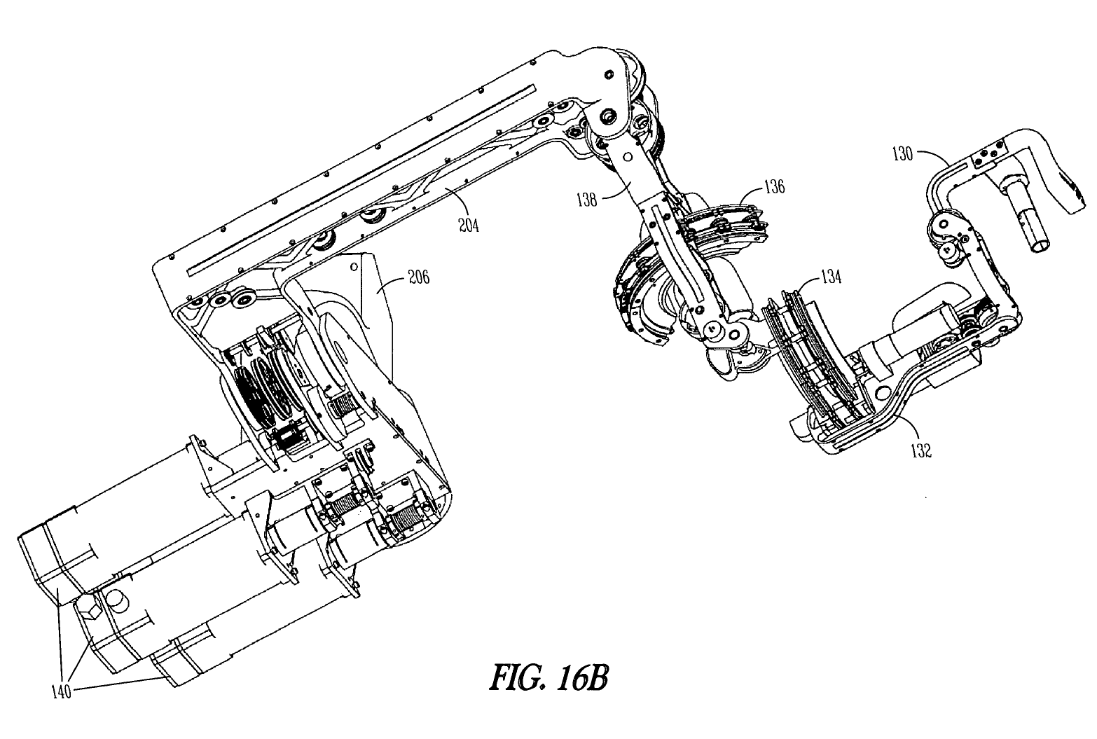

FIGS. 16A and 16B illustrate two different perspective views of an exoskeleton where cables are removed from the power drive for the sake of image clarity. The exoskeleton illustration includes hand piece 130, lower arm link 132, circular bearing 134 for the lower arm, circular bearing 136 for the upper arm, upper arm link 138, and actuators 140. The figures also illustrate attachment bracket 206 (for attachment to a stable platform), and connecting link 204.

The above description is intended to be illustrative, and not restrictive. For example, the above-described embodiments (or one or more aspects thereof) may be used in combination with each other. Other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to comply with 37 C.F.R. §1.72(b), which requires that it allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment.

What is claimed is:

1. A system comprising:
an upper link member coupled to a support frame at a shoulder joint;
a lower link member coupled to the upper link member at an elbow joint;
a hand member coupled to the lower link member at a wrist joint;
a plurality of transducers affixed to at least one of the upper link member, the lower link member, and the hand member;
a driver coupled to the frame and at least one of the upper link member, the lower link member and the hand member and wherein the driver is configured to control at least one of position and velocity relative to the frame; and
a processor configured to execute instructions to control the driver based on a signal received from at least one transducer of the plurality of transducers; and
wherein the upper link member and the lower link member are configured for attachment to an arm of a user and wherein a rotation axis of each of the shoulder joint, the elbow joint and the wrist joint are aligned with corresponding axes of the user.

2. The system of claim 1 wherein the support frame includes at least one of a wheelchair and a stationary structure.

3. The system of claim 1 wherein at least one of the upper link member and the lower link member are aligned with a portion of the arm.

4. The system of claim 1 wherein the shoulder joint has three degrees of freedom.

5. The system of claim 1 wherein the elbow joint has two degrees of freedom.

6. The system of claim 1 wherein the wrist joint has two degrees of freedom.

7. The system of claim 1 wherein the plurality of transducers includes at least one of an image sensor, a strain gauge, a force-torque sensor, an accelerometer, and an encoder.

8. The system of claim 1 wherein the driver includes at least one of a motor and a cable operated transmission.

9. The system of claim 1 wherein the processor is configured to control the driver in a simulated environment.

10. The system of claim 1 wherein the processor is configured to control the driver to exert feedback to the arm.

11. The system of claim 1 wherein at least one transducer is configured to generate an output signal based on a neural signal detected at a surface of the user.

12. A method comprising:
generating an image of a scene;
receiving information from a transducer of a wearable exoskeleton, wherein the wearable exoskeleton includes a plurality of links, each link having an articulating joint, wherein each joint is aligned with an axis of an anatomical joint, the information corresponding to a simulated limb interacting in the scene; and
modifying performance of the simulated limb based on an element in the scene.

13. The method of claim 12 wherein the scene is a virtual scene.

14. The method of claim 12 wherein generating the image of a scene includes receiving information over a communication channel.

15. The method of claim 12 wherein receiving information from the transducer includes receiving a signal from a surface sensor coupled to a user.

16. The method of claim 12 wherein modifying performance includes limiting a range of motion of a link of the plurality of links.

17. The method of claim 12 wherein modifying performance includes exerting a resistive force or torque to an articulating joint.

18. A method comprising:
coupling a user to an exoskeleton having a plurality of exoskeleton links and a plurality of exoskeleton joints, the plurality of exoskeleton links corresponding to an upper limb of the user and each of the plurality of exoskeleton joints corresponding to an anatomical joint of the upper limb of the user and wherein the axis of each joint of the exoskeleton is aligned with a corresponding anatomical joint of the user;
receiving a feedback signal from a surface sensor coupled to the user;
executing an algorithm to determine a torque for at least one exoskeleton joint based on the feedback signal; and
applying the torque to the least one exoskeleton joint.

19. The method of claim 18 wherein coupling the user to the exoskeleton includes attaching the exoskeleton to at least one of an upper forearm of the user, a lower forearm of the user, and a hand of the user.

20. The method of claim 18 wherein applying the torque includes applying an assistive torque.

21. The method of claim 18 wherein applying the torque includes applying a resistive torque.

22. The method of claim 18 wherein executing the algorithm includes executing an algorithm to increase a range of motion.

23. The method of claim 18 wherein executing the algorithm includes executing an algorithm to resist movement of an anatomical joint.

24. The method of claim 18 wherein applying the torque includes controlling a motor drive.

25. The method of claim 18 wherein applying the torque includes operating a brake.
